(19) United States
(12) Patent Application Publication
(10) Pub. No.: US 2025/0349139 A1
(43) Pub. Date: Nov. 13, 2025

(54) IMAGE RECOGNITION PROGRAM, IMAGE RECOGNITION DEVICE USING SAME, DETECTION TARGET NUMBER COUNTING METHOD, AND MODEL IMAGE CREATION DEVICE FOR IMAGE RECOGNITION TRAINING USED THEREFOR

(71) Applicant: NATIONAL INSTITUTES FOR QUANTUM SCIENCE AND TECHNOLOGY, Chiba-shi, Chiba (JP)

(72) Inventors: Shinichiro MORI, Chiba-shi, Chiba (JP); Michiyo SUZUKI, Chiba-shi, Chiba (JP); Yoshinobu HARADA, Chiba-shi, Chiba (JP); Yasuhiko TACHIBANA, Chiba-shi, Chiba (JP); Yuya HATTORI, Chiba-shi, Chiba (JP)

(21) Appl. No.: 18/847,954

(22) PCT Filed: Feb. 27, 2023
(86) PCT No.: PCT/JP2023/007082

§ 371 Date: Jul. 31, 2025

(30) Foreign Application Priority Data
Mar. 17, 2022 (JP) 2022-042659

(51) Int. Cl. G06V 20/69; G06V 10/77; G06V 10/80; G06V 10/82

(52) U.S. Cl. CPC G06V 20/698; G06V 10/7715; G06V 10/80; G06V 10/82; G06V 2201/07

(57) ABSTRACT

Provided are an image recognition program capable of accurately counting the population of nematodes that are detection targets even when a plurality of detection targets overlap each other, an image recognition device using the same, a detection target population counting method, and a model image creation device for image recognition learning to be used therefor.

An image recognition program causes a control unit 11 and a storage unit 12 of an image recognition device 1 to function as detection target image acquisition means for acquiring a detection target image showing a plurality of nematodes 3, extraction means for extracting from the detection target image a detection target presence area that possibly includes an image of the nematodes 3, storage means for storing a plurality of pattern images including a single-body pattern image showing one of the nematodes 3 and a multiple-bodies pattern image corresponding to an image showing two or more of the nematodes 3 overlapping each other, and recognition means for recognizing the population of nematodes 3 included in the detection target presence area by detecting a degree of concordance between an image of the detection target presence area and each of the pattern images.

### TECHNICAL FIELD

This invention relates to an image recognition program for counting the population of detection targets from a detection target image showing a plurality of detection targets, an image recognition device using the same, a detection target population counting method, and a model image creation device for image recognition learning to be used therefor.

### BACKGROUND TECHNOLOGY

The nematode Caenorhabditis elegans (sometimes referred to as “C. elegans”) is an established model organism for research in neurobiology, developmental biology, and gerontology. In particular, in the field of neurobiology, assays based on a taxis assay method established in the 1990s (e.g., Non Patent Literature 1, sometimes referred to as the “first taxis assay method”) have been conducted to investigate the response (taxis) of the nematode C. elegans to chemicals and the like. In the first taxis assay method, nematodes are released onto a solid medium on which a gradient of the assay target substance or temperature is formed, and the response of the nematodes is evaluated by counting the individuals that approach the target substance and the individuals that escape. This method requires counting populations of nematodes that are extremely small compared to the size of the assay plate, and there are problems such as the low efficiency of the assay due to the large amount of labor required for counting and the use of anesthetics to trap the nematodes.

In response to the above issues, a nematode trap plate that significantly improves on the first taxis assay method and a new taxis assay method using the same (sometimes referred to as the “second taxis assay method”) were proposed in 2019 (Patent Literature 1). In the second taxis assay method, two or more recesses that are extremely small compared to the size of the assay plate are formed and filled with a test liquid and a control standard solution, and the population of nematodes that are attracted to the test substance or the like and trapped in the recesses, or that escape from the test substance and are trapped in the control recesses, is counted.

The first taxis assay method requires counting populations of nematodes crawling on a solid medium, while the second taxis assay method requires counting populations of nematodes swimming in a liquid recess. In particular, since multiple nematodes overlap in a liquid recess and repeat flexion and extension at high speed (swimming motion), it is difficult to accurately count the population of nematodes using conventional nematode population counting methods, and there is room for improvement in terms of accuracy.

### CITATION LIST

### Non Patent Literature

Non Patent Literature 1: Bargmann, C. I., et al., Odorant-selective genes and neurons mediate olfaction in C. elegans. Cell, 74, 515-527, 1993.

### Patent Literature

Patent Literature 1: International Publication No. 2020/218501.

### SUMMARY OF INVENTION

### Technical Problem

In view of the above-mentioned problems, an object of this invention is to provide an image recognition program capable of accurately counting the population of detection targets even when a plurality of detection targets overlap each other in a detection target image showing the plurality of detection targets, an image recognition device using the same, a detection target population counting method, and a model image creation device for image recognition learning to be used therefor.

### Solution to Problem

This invention is characterized by an image recognition program for causing a computer to function as: detection target image acquisition means for acquiring a detection target image showing a plurality of detection targets; extraction means for extracting from the detection target image a detection target presence area that possibly includes an image of the detection targets; storage means for storing a plurality of pattern images including a single-body pattern image showing one of the detection targets and a multiple-bodies pattern image corresponding to an image showing two or more of the detection targets overlapping each other; and recognition means for recognizing the population of detection targets included in the detection target presence area by detecting a degree of concordance between an image of the detection target presence area and each of the pattern images.

### Advantageous Effects of Invention

According to this invention, it is possible to accurately count the population of detection targets even when a plurality of detection targets overlap each other in a detection target image showing the plurality of detection targets.

### BRIEF DESCRIPTION OF DRAWINGS

FIG. 1 is a block diagram showing the configuration of an image recognition device.

FIG. 2 is a top view showing an example of a nematode trap plate.

FIG. 3 is an A-A cross-sectional view of the nematode trap plate.

FIG. 4 is a flowchart showing an image acquisition process in which the image recognition device acquires the detection target image.

FIG. 5 is an enlarged view of the nematode trapping recess filled with test liquid.

FIG. 6 shows an example of an unprocessed detection target image acquired by an image recognition device.

FIG. 7a shows an example of an image of swimming nematodes captured 0.1 seconds after observation began.

FIG. 7b shows an example of an image of swimming nematodes captured 0.2 seconds after observation began.

FIG. 7c shows an example of an image of swimming nematodes captured 0.3 seconds after observation began.

FIG. 8 is a diagram showing an example of two single-body pattern images, each of which shows a single nematode.

FIG. 9a is a diagram showing an example of a label “2” pattern image showing two overlapping nematodes.

FIG. 9b is a diagram showing an example of a label “3” pattern image showing three overlapping nematodes.

FIG. 9c is a diagram showing an example of a label “4” pattern image showing four overlapping nematodes.

FIG. 10 is a flowchart showing a multiple-bodies pattern image creation process in which an image recognition device creates a multiple-bodies pattern image.

FIG. 11a is a diagram showing an example of a processed single-body pattern image.

FIG. 11b is a diagram showing an example of a multiple-bodies pattern image formed by overlaying processed single-body pattern images.

FIG. 12 is a flowchart showing a model image creation process in which an image recognition device creates a model image for learning.

FIG. 13 is a diagram showing an example of a model image created by an image recognition device.

FIG. 14 is a flowchart showing a learning process in which an image recognition device learns using a model image.

FIG. 15 is a flowchart showing a detection target population counting process in which an image recognition device acquires a detection target image and counts the population of detection targets.

FIG. 16 is a diagram showing an example of an image indicating the image recognition result of the detection target.

FIG. 17a shows an example of an unprocessed detection target image captured with a suitable amount of liquid and under suitable lighting.

FIG. 17b shows an example of an unprocessed detection target image with image quality when only a small portion of the nematodes in the liquid is in focus.

FIG. 17c shows an example of an unprocessed detection target image in which nematodes in a liquid (test liquid) containing a biological sample mixed with impurities are captured.

FIG. 17d shows an example of an unprocessed detection target image in which nematodes in a turbid liquid (test liquid) containing a biological sample are captured.

FIG. 17e shows an example of an unprocessed detection target image with strong shadows on the edge of the liquid recess.

FIG. 17f shows an example of an unprocessed detection target image with image quality that shows a lot of reflected light from the surface of the liquid recess.

### DESCRIPTION OF EMBODIMENTS

An embodiment of the present invention will be described below with reference to the drawings.

### Image Recognition Device

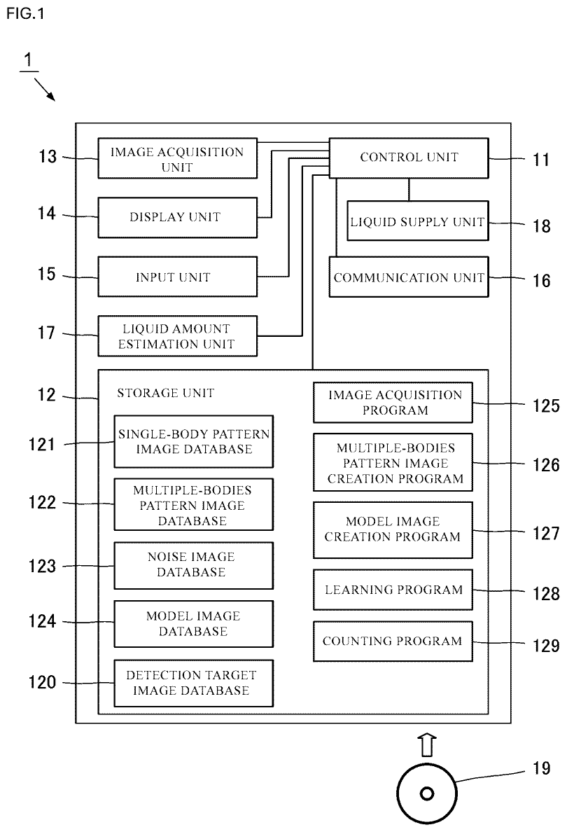

FIG. 1 is a block diagram showing the configuration of an image recognition device 1 (detection target population counting device).

The image recognition device 1 is a device for counting the population of detection targets from a detection target image showing a plurality of detection targets. In this specification, the term “detection target image” refers to both an “unprocessed detection target image” that is the taken (captured) photographic image that has not been processed, i.e., a primary image, and a “processed detection target image” obtained by performing, on the unprocessed detection target image, appropriate image processing such as processing to remove unnecessary parts such as edges and processing to make the background shading uniform, and indicates that it can be either an “unprocessed detection target image” or a “processed detection target image”. The image recognition device 1 is a computer terminal equipped with the following hardware elements: a control unit 11 that is composed of a CPU, a ROM, a RAM, etc. and executes various calculations and control operations; a storage unit 12 that is composed of a hard disk, a flash memory, etc. and allows reading and writing of information; an image acquisition unit 13 that is composed of a camera, etc. and acquires images; a display unit 14 that is composed of a liquid crystal display, an organic EL display, etc. and displays images such as characters and figures; an input unit 15 that is composed of a touch panel, a keyboard, a computer mouse (pointing device), a touch pen, a push button, or a combination of these and accepts input by a touch operation; a communication unit 16 that is composed of a LAN board, a WiFi unit, etc. and transmits and receives data; a liquid amount estimation unit 17 that estimates an amount of the liquid in the liquid recess or an amount of the liquid with which the liquid recess is refilled (supplied); and a liquid supply unit 18 that refills (supplies) the liquid recess with the liquid by the amount estimated by the liquid amount estimation unit 17. Each of the storage unit 12, the image acquisition unit 13, the display unit 14, the input unit 15, and the communication unit 16 is connected to the control unit 11 via a communication line (bus).

The control unit 11 includes a calculation unit and a main storage unit, and executes various calculations and control operations in the image recognition device 1. The calculation unit is a calculation processing unit including a CPU or MPU. The main storage unit has RAM (DRAM) and ROM. The RAM is used as a work area and buffer area for the calculation unit. The ROM stores the startup program for the image recognition device 1 and default values for various information.

The image acquisition unit 13 is composed of, for example, a lens and a shutter for taking pictures with the camera. The shutter is controlled by the control unit 11. When the shutter is released, the image acquisition unit 13 acquires an image of the subject reflected by the lens at that time. The image acquisition unit 13 also has a zoom function for enlarging or reducing the subject reflected by the lens, and a focus correction function for focusing on the subject. The zoom function and focus correction function are controlled by the control unit 11. In this embodiment, the image acquisition unit 13 includes a fluorescent microscope equipped with a magnifying lens of a predetermined magnification. The shutter may be operated by the user of the image recognition device 1. The camera of the image acquisition unit 13 may be a high-speed camera.

The display unit 14 has a display and a display control circuit interposed between the display and the calculation unit. The display may be, for example, an LCD (liquid crystal display) or an organic EL display. The display control circuit has a GPU and a VRAM. Under the direction of the calculation unit, the GPU uses image generation data stored in the RAM to generate display image data in the VRAM for displaying various screens (such as the registration screen 100 described below) on the display, and outputs the generated display image data to the display.

The input unit 15 has input components that accept operational input from the user of the image recognition device 1, and an input detection circuit that is interposed between the input components and the calculation unit. The input components are, for example, a touch panel (touch input means) and/or hardware operation buttons or operation keys. The input components may also include a computer mouse or the like as a pointer input means. The touch panel may be of any type, such as a capacitive type, an electromagnetic induction type, a resistive film type, or an infrared type. The input detection circuit outputs an operation signal or operation data corresponding to the operation (operation input) of each input component to the calculation unit.

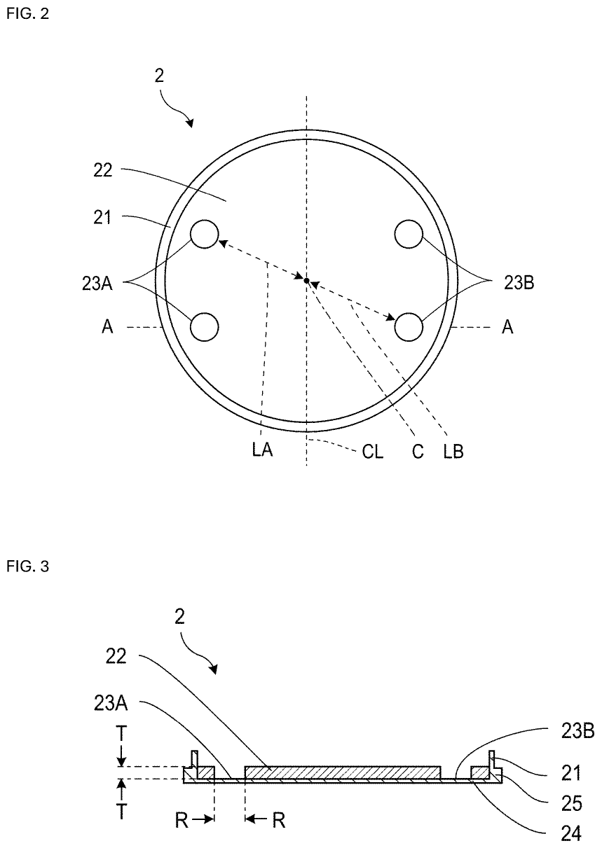

The liquid amount estimation unit 17 is, for example, a timer controlled by the control unit 11, which measures the elapsed time from a start time to an end time when a certain time is set as the start time and a time further in the future is set as the end time. In the case of this timer, the liquid amount estimation unit 17 calculates the amount of evaporation of the liquid in the liquid recess and the amount absorbed by the solid medium based on the elapsed time, and calculates how much liquid has decreased. The calculated amount of the liquid is then estimated as the amount of the liquid with which the liquid recess is refilled (amount of refill liquid). Alternatively, the liquid amount estimation unit 17 may be, for example, a camera controlled by the control unit 11 and composed of a lens and a shutter for taking pictures. In this case, the amount of the liquid in the nematode trapping recesses (nematode trapping recesses 23A and 23B, hereinafter sometimes simply referred to as “trapping recesses”) (see FIG. 2) provided in the nematode trap plate 2 is confirmed by photographing from an oblique direction or from above, and the amount of the liquid required (amount of refill liquid) is estimated to bring the amount of the liquid into the state in which the height of the liquid level 234 (see FIG. 5, FIG. 5 is an example of trapping recess 23A) of the liquid in the trapping recesses 23A and 23B after refilling the liquid falls within a predetermined range (allowable range S) including the position of the upper edge 233 of the peripheral wall 232 of the trapping recesses 23A and 23B. Alternatively, the liquid amount estimation unit 17 may be configured, for example, as a range finder controlled by the control unit 11 that irradiates infrared light or the like and reads the reflected light. In this case, the amount of the liquid in the trapping recesses 23A and 23B is confirmed by measurement from above, and the amount of the liquid required (amount of refill liquid) is estimated to bring the amount of the liquid into the state adjusted for image capture in which the height of the liquid level 234 of the liquid in the trapping recesses 23A and 23B after refilling the liquid falls within a predetermined range (allowable range S) that includes the position of the upper edge 233 of the peripheral wall 232 of the trapping recesses 23A and 23B.

The liquid supply unit 18 is, for example, a dropper or pipette that is controlled by the control unit 11 and drips pre-filled liquid into the nematode trapping recesses 23A and 23B. The liquid supply unit 18 refills the liquid recess with the liquid by the insufficient amount (amount of refill liquid) calculated by the liquid amount estimation unit 17 under the control of the control unit 11.

The storage unit 12 includes a single-body pattern image database 121 which is a database of image data (single-body pattern image) showing a single detection target, a multiple-bodies pattern image database 122 which is a database of image data (multiple-bodies pattern image) showing a plurality of detection targets, a noise image database 123 which is a database of image data showing noise other than the detection targets, a model image database 124 which is a database of image data (model image data) for machine learning created by combining a single-body pattern image, a multiple-bodies pattern image, and a noise image, and a detection target image database 120 which is a database of image data (detection target image) showing the plurality of detection targets. Note that a single-body pattern image is image data including an image of one detection target, and a multiple-bodies pattern image is image data including images of two or more detection targets. That is, in this embodiment, a single-body pattern image is an image showing one nematode (detection target), and a multiple-bodies pattern image is an image showing two or more nematodes. The storage unit 12 also stores an image acquisition program 125 that the control unit 11 executes to acquire a detection target image, a multiple-bodies pattern image creation program 126 that the control unit 11 executes to create a multiple-bodies pattern image, a model image creation program 127 that the control unit 11 executes to create a model image, a learning program 128 that the control unit 11 uses the model image to learn about the detection target, and a counting program 129 (detection target population counting program) that the control unit 11 uses to count the population of detection targets in the target image.

### Nematode Trap Plate

FIG. 2 is a top view showing an example of the nematode trap plate 2, and FIG. 3 is a cross-sectional view captured along the line A-A of the nematode trap plate 2. Below, a configuration for obtaining image data showing the detection target will be described with reference to FIGS. 2 and 3. Note that this nematode trap plate 2 and the above-mentioned image recognition device 1 (including a camera) constitute a detection target population counting system that counts the population of detection targets.

In this embodiment, the detection target was the nematode. In the present invention, nematodes refer to both animals belonging to the phylum Nematoda and animals belonging to the phylum Nematomorpha in biological taxonomy. There are no particular limitations on the nematodes that can be the target of detection, as long as they are terrestrial or semi-terrestrial and can move on a solid phase among the animals included in the above. In the present invention, the term “nematode” includes nematodes in each developmental stage. Nematodes in each developmental stage include nematode eggs (including fertilized eggs), larvae (1st to 4th instars), and adults. Adult nematodes include nematodes of each sex. Nematodes of each sex include male, female, and hermaphrodite nematodes. In this embodiment, it is assumed that nematodes are used that have the property of being attracted to or repelled by environmental stimuli such as a specific water-soluble substance (taste), a specific volatile substance (smell), or a specific temperature.

Animals belonging to the phylum Nematoda include various kinds of nematodes, such as nonparasitic nematodes (or free-living nematodes), plant-parasitic nematodes, entomogenous nematodes (including entomopathogenic nematodes, parasitoid nematodes, and entomoparasitic nematodes), nematodes parasitic on insects, etc., and nematodes parasitic on mammals, etc.

Examples of nonparasitic nematodes include Caenorhabditis elegans (hereinafter sometimes referred to as “C. elegans”), Aphelenchus avenae, Caenorhabditis angaria, Caenorhabditis brenneri, Caenorhabditis briggsae, Caenorhabditis japonica, Caenorhabditis remanei, and Pristionchus pacificus.

Examples of plant-parasitic nematodes include Acreberoides nanus, Bastiania gracilis, Wilsonema othophorum, Meloidogyne incognita, Meloidogyne arenaria, Meloidogyne javanica, Meloidogyne hapla, Meloidogyne marytlandi, Meloidogyne mali, Heterodera glycines, Heterodera schachtii, Heterodera elachista, Globodera rostochiensis, Globodera pallida, Hirschmanniella diversa, Hirschmanniella immamuri, Pratylenchus penetrans, Pratylenchus coffeae, Pratylenchus vulnus, Pratylenchus neglectus, Pratylenchus loosi Loof, Pratylenchus curvitatus, Pratylenchus kumamotoensis, Pratylenchus pseudocoffeae, Ditylenchus dipsaci, Helicotylenchus dihystera, Helicotylenchus erythrinae, Hemicriconemoides kanayaensis, Paratrichodorus mirzai, Paratrichodorus minor (Colbran) Siddiqi or Trichodorus minor Colbran, Trichodorus porosus, Pratylenchus zeae Graham, Tetraopes annulatus, Tylenchorhynchus nudus, Aphelenchoides besseyi, Anguina tritici, Ditylenchus destractor, Aphelenchoides ritzemabosi, Longidorus spp., Xiphinema index, Aphelenchoides fragariae, Nothotylenchus acris, Pratylenchus brachyurus, Radopholus similis, Caenorhabditis inopinata, and Xiphinema brevicolle.

Examples of entomoparasitic nematodes include Sphaerularia bombi, Sphaerularia vespae, Agamermis unka, Amphimermis zuimushi, Hexamermis microamphidis, Steinernema carpocapsae, Steinernema kushidai, Iotonchium ungulatum, Iotonchium californicum, Iotonchium cateniforme, Iotonchium laccariae, Iotonchium russulae, Caenorhabditis auriculariae, Bursaphelenchus tadamiensis, Contortylenchus sp., Contortylenchus genitalicola, and Romanomermis culicivorax.

Examples of phoretic nematodes parasitic on insects, etc. include Caenorhabditis japonica, Pristionchus pacificus, Bursaphelenchus xylophilus, Bursaphelenchus mucronatus, Bursaphelenchus doui, Bursaphelenchus firmae, Bursaphelenchus conicaudatus, Bursaphelenchus luxuriosae, Bursaphelenchus hunti, Bursaphelenchus okinawaensis, Bursaphelenchus yongensis, Bursaphelenchus kiyoharai, Bursaphelenchus cocophilus, Bursaphelenchus niphades, Bursaphelenchus sexdentati, Teratorhabditis synpapillata, Caenorhabditis briggsae, and Caenorhabditis remane.

Examples of the nematodes parasitic on mammals, etc. include Strongyloides stercoralis, filariae, Ascaridida, Anisakis, whipworms, hookworms, Gnathostoma spp., and Trichinella spp.

Examples of the Strongyloides stercoralis are classified according to each taxonomic order of the primary host animals: Strongyloides stercoralis parasitic on animals of the order Anura of the class Amphibia (Strongyloides pereira (hereinafter, the generic name of the Strongyloides stercoralis, “Strongyloides”, may be abbreviated simply as “S.”), S. carinii, S. amphibiophilus, S. bufonis, S. physali, S. spiralis, S. prokopici, S. mascomai, etc.), Strongyloides stercoralis parasitic on animals of the order Lacertilia of the class Reptilia (S. cruzi, S. darevskyi, S. ophiusensis, etc.), Strongyloides stercoralis parasitic on animals of the order Serpentes of the class Reptilia (S. ophidiae, S. mirzai, S. gulae, S. serpentis, etc.), Strongyloides stercoralis parasitic on animals of the order Ciconiiformes of the class Aves (S. cubaensis, S. ardeae, S. herodiae, etc.), Strongyloides stercoralis parasitic on animals of the order Galliformes of the class Aves (S. avium, S. oswaldoi, and S. pavonis, etc.), Strongyloides stercoralis parasitic on animals of the order Anseriformes of the class Aves (S. minimum, etc.), Strongyloides stercoralis parasitic on animals of the order Charadriiformes of the class Aves (S. turkmenica, etc.), Strongyloides stercoralis parasitic on animals of the order Passeriformes of the class Aves (S. quiscali Barus, etc.), Strongyloides stercoralis parasitic on animals of the order Marsupialia of the class Mammalia (S. thylacis, etc.), Strongyloides stercoralis parasitic on animals of the order Insectivora of the class Mammalia (S. akbari, and S. rostombekowi, etc.), Strongyloides stercoralis parasitic on animals of the order Primates of the class Mammalia (S. stercoralis, S. fuelleborni, S. fuelleborni kellyi, and S. cebus, etc.), Strongyloides stercoralis parasitic on animals of the order Xenarthra of the class Mammalia (S. dasypodis, and S. shastensis, etc.), Strongyloides stercoralis parasitic on animals of the Pholidota of the class Mammalia (S. leiperi, etc.), Strongyloides stercoralis parasitic on animals of the order Rodentia of the class Mammalia (S. chapini, S. ratti, S. myopotami, S. venezuelensis, S. agoutii, S. robustus, and S. sigmodontis, etc.), Strongyloides stercoralis parasitic on animals of the order Carnivora of the class Mammalia (S. nasua, S. felis, S. mustelorum, S. erschowi, S. planiceps, S. puttori, S. martis, S. vulpis, S. tumefasciens, S. lutrae, and S. procyonis, etc.), Strongyloides stercoralis parasitic on animals of the order Proboscidea of the class Mammalia (S. elephantis, etc.), Strongyloides stercoralis parasitic on animals of the order Perissodactyla of the class Mammalia (S. westeri, etc.), and Strongyloides stercoralis parasitic on animals of the order Artiodactyla of the class Mammalia (S. papillosus, S. ransomi, etc.).

Examples of filariae include Parafilaria multipapillosa, Stephanofilaria okinawaensis, Wuchereria bancrofti, Brugia malayi, Onchocerca cervicalis, Onchocerca gibsoni, Onchocerca gutturosa, Onchocerca volvulus, Acanthocheilonema reconditum, Setaria digitata, Setaria equina, Setaria labiatopapillosa, Setaria marshalli, Dirofilaria immitis, and Loa loa.

Examples of Ascaridida include Ascaris lumbricoides, Ascaris suum, Baylisascaris transfuga, Lagochilascaris minor, Toxocara vitulorum (or Neoascaris vitulorum), Parascaris equorum, Baylisascaris procyonis, Toxocara canis, Toxocara cati, Toxascaris leonina, and Toxocara tanuki.

Examples of Anisakis include so-called Anisakis type I, such as Anisakis pegreffii, Anisakis simplex sensu stricto, and Anisakis simplex C, Anisakis type II (Anisakis physeteris), Psudoterranova decipiens, and Contracaecum osculatum.

Examples of whipworms include Trichuris discolor, Trichuris muris, Trichuris ovis, Trichuris suis, Trichuris trichiura, and Trichuris vulpis.

Examples of hookworms include Ancylostoma braziliense, Ancylostoma caninum, Ancylostoma ceylanicum, Ancylostoma duodenale, Ancylostoma kusimaense, Ancylostoma malayanum, Arthrostoma miyazakiense, Ancylostoma tubaeforme, Uncinaria stenocephala, Bunostomum phlebotomum, Bunostomum trigonocephalum, Globocepharus urosubulatus, and Necator americanus.

Examples of Gnathostoma spp. include Gnathostoma nipponicum, Gnathostoma spinigerum, Gnathostoma hispidum, Gnathostoma doloresi, Gnathostoma procyonis, and Gnathostoma vietnamicum.

Examples of Trichinella spp. include Trichinella britovi, Trichinella spiralis, Trichinella nativa, Trichinella nelsoni, and Trichinella pseudospiralis.

Examples of nematodes parasitic on mammals etc. other than those mentioned above include the Abbreviata caucasica, Physaloptera praeputialis, Thelazia callipaeda, Thelazia rhodesi, Thelazia skrjabini, Habronema microstoma, Habronema muscae, Draschia megastoma, Crassicauda giliakiana, Gongylonema pulchrum, Ascarops strongylina, Physocephalus sexalatus, etc. Other examples include: strongyles such as Strongylus asini, Strongylus edentates, Strongylus equinus, and Strongylus vulgaris, as well as Capillaria (also called capillary nematodes) such as Eucoleus annulate, Eucoleus contorta, Pearsonema feliscati, Eucoleus perforans, Paracapillaria philippinensis (or Capillaria philippinensis), Calodium hepatica (or Capillaria aerophila), Aonchotheca bovis, Eucoleus aerophile, and Pearsonema plica. Furthermore, the examples include trichostrongyle nematodes such as Trichostrongylus axei, Trichostrongylus colubriformis, and Trichostrongylus orientalis, twisted stomach worms such as Haemonchus contortus and Mecistocirrus digitatus, Dictyocaulus filaria, Dictyocaulus viviparus, Nematodirus filicollis, Angiostrongylus such as Angiostrongylus cantonensis and Angiostrongylus costaricensis, etc.; Metastrongylus elongatus, Filaroides hirthi, and the like. Further examples include pinworms such as Enterobius vermicularis, Passalurus ambiguous, Syphacia muris, Syphacia obvelata, and Oxyuris equi.

Furthermore, examples of animals belonging to the phylum Nematomorpha include horsehair worms (Gordioidea). Examples of Gordioidea include Gordius robustus, Gordius ogatai, Pseudogordius tanganykae, and Chordodes japonensis.

In this embodiment, the detection target was Caenorhabditis elegans (C. elegans).

In this embodiment, a nematode trap plate with recesses for trapping nematodes was used. In this embodiment, a nematode trap plate 2 having two nematode trapping recesses on each of the left and right sides of the plate will be used as an example. The nematode trap plate 2 is composed of a container 21, a solid phase 22 formed in the container 21, and the nematode trapping recesses 23A and 23B provided in the solid phase 22.

The container 21 is a box-shaped container with an open top, having a bottom wall portion 24 and a peripheral wall 25 rising from the edge of the bottom wall portion 24. The peripheral wall 25 may have a polygonal or circular cross section. In this embodiment, the peripheral wall is formed into a cylindrical shape with a circular cross section. The container 21 is made of a transparent material, and is hard enough that it is not deformed by at least a human hand. For example, glass or plastic can be used as a material for the container 21, and the container 21 can be a glass-based dish or a plastic dish. As the plastic, synthetic resins in general, including acrylic resins, can be used. The solid phase 22 is formed inside the container 21.

The solid phase 22 is intended to be a solid layer formed in the container 21, and is not particularly limited as long as the nematodes can move on its surface (upper surface). Specifically, the solid phase 22 is assumed to be a layer that contains water and has a wet surface. Non-limiting examples of the solid layer include gels formed from agar, agarose, gelatin, konjac, etc., as well as gels formed by adding additives such as gelling agents or thickening stabilizers, such as pectin, guar gum, carrageenan, and xanthan gum, to a liquid. The solid phase 22 is preferably a solid medium solidified or gelled with agar or the like. This ensures smooth movement of the nematodes on the solid phase 22. The solid phase 22 is also preferably a solid medium formed by solidifying or gelling a natural raw material such as agar. These are solid phases that do not inhibit the biological characteristics of the nematodes, that is, have good biocompatibility. Furthermore, the solid phase 22 is preferably tasteless and odorless so as not to affect the response of nematodes in an assay using a nematode trap plate. A sulfur source, phosphate, and a trace amount of minerals can also be added to the solid medium used as the solid phase 22. For example, one or more of magnesium sulfate (MgSO₄), potassium dihydrogen phosphate (KH₂PO₄), dipotassium hydrogen phosphate (K₂HPO₄), and calcium chloride (CaCl₂) may be added. The solid phase 22 may be, for example, a medium obtained by gelling or solidifying a medium for living organisms. In this embodiment, a solid medium obtained by solidifying a medium for living organisms was used as the solid phase 22. In addition, the solid phase 22 has the nematode trapping recesses 23A and 23B formed therein.

Two nematode trapping recesses 23A are formed near the end of the solid phase 22. Two nematode trapping recesses 23B are formed near the end opposite to the position of the trapping recess 23A. The number of trapping recesses 23A and 23B is not limited to this. For example, one may be formed near the end of the solid phase 22 and another near the opposite end, or one may be formed near the end of the solid phase 22 and none may be formed near the opposite end. The distance LA from the center position (center position) C of the solid phase 22 to the trapping recess 23A is the same as the distance LB from the center position C to the trapping recess 23B. In this embodiment, the trapping recesses 23A and the trapping recess 23B are arranged symmetrically about an arbitrary line passing through the center position C of the upper surface of the solid phase 22 when the nematode trap plate 2 is viewed from above. The trapping recesses 23A and 23B are formed to extend from the surface of the solid phase 22 toward the bottom of the solid phase 22. The trapping recesses 23A and 23B have a cylindrical shape with a diameter R and a depth T. The diameter R of the trapping recesses 23A and 23B can be adjusted appropriately depending on the population and size of the nematodes. In this embodiment, the diameter R was set to 5 mm. The depth T of the trapping recesses 23A and 23B can be set to 1 to 10 mm, and is preferably set to 2 to 3 mm. In this embodiment, the depth T was set to 2 mm.

### Nematode Trapping

The adult nematode C. elegans uses its long, slender body, approximately 1 mm long and 60 μm wide, to perform high-speed periodic movements that constantly repeat flexion and extension. Nematodes also have the property of being attracted or repelled by certain volatile substances (chemotaxis). When investigating whether nematodes are attracted to or repelled by a certain substance, the nematode trapping recesses 23A (23B) provided on the nematode trap plate 2 can be used as a test liquid recess filled with a liquid (test liquid) containing the substance to be tested, and the other nematode trapping recesses 23B (23A) can be used as a standard liquid recess filled with a control liquid (standard liquid). This makes it possible to investigate the chemotaxis of the nematode to the test substance. The other control liquid can be an appropriate liquid that serves as a control for the one liquid, such as a liquid to which the nematodes do not react.

In this embodiment, the two nematode trapping recesses 23A were filled with a liquid (test liquid) containing the substance to be tested to form a test liquid recess. The test liquid may be a diluted solution of a volatile (scented) substance such as diacetyl, or a liquid containing a body fluid such as animal urine or blood. The remaining two control nematode trapping recesses 23B were filled with a liquid that would not affect the nematode taxis assay to form a standard liquid recess. As such a liquid, the solvent used to dilute the test substance may be used, and water, physiological saline, ultrapure water, and buffer solutions commonly used in nematode experiments may be used. After that, approximately 100 adult nematodes were supplied near the central position C of the solid phase 22, and after one hour, the two trapping recesses 23A and the two trapping recesses 23B were photographed to obtain unprocessed detection target images. For example, when the test liquid was a low concentration dilution of diacetyl, which is preferred by nematodes, approximately 100 nematodes were attracted to the test liquid and trapped in one of the two trapping recesses 23A, and almost no nematodes were trapped in the two trapping recesses 23B on the control side. On the other hand, when the test liquid was a high concentration dilution of diacetyl, which nematodes dislike, all nematodes escaped from the two trapping recesses 23A filled with the test liquid, and approximately 100 nematodes were trapped in one of the two trapping recesses 23B on the control side. In this way, whether the nematodes are trapped in the trapping recesses 23A or the trapping recesses 23B depends on the type of test liquid filled in the test liquid recess. The test liquid recess and the control standard liquid recess are each formed by a recess that is deep enough for the body length of the nematodes, so that the nematodes that once entered the test liquid recess or the standard liquid recess will remain in the recess. In the chemotaxis assay using the nematode trap plate 2 (the chemotaxis assay based on the second chemotaxis assay method), the population of nematodes trapped in the trapping recess 23A and the trapping recess 23B is counted, and the chemotaxis is evaluated based on the counted population. From the viewpoint of counting the population of nematodes by image recognition, 0 to 100 nematodes are present per 0.01 mm² of the opening area of the trapping recess 23A and the trapping recess 23B when viewed from the upper side of the trapping recess 23A and the trapping recess 23B. The nematodes referred to here include nematode eggs (including fertilized eggs), larvae (1st to 4th instars), and nematodes in each developmental stage (stage) of adults. In addition, it is assumed that two or more nematodes are trapped in the trapping recess 23A or the trapping recess 23B. When the diameter R of the trapping recess 23A and the trapping recess 23B is 5 mm and the depth T is 2 mm (when the volume (capacity) of the test liquid recess and the standard liquid recess is approximately 40 mm³), the number of nematodes trapped in the trapping recess 23A can be set to 2 or more at the lower limit, preferably 10 or more, and 200 or less at the upper limit, preferably 100 or less, when using adult nematodes. Therefore, when using the nematode trap plate 2 of this embodiment, the number of nematodes supplied can be set to 4 or more at the lower limit, preferably 20 or more, and 300 or less at the upper limit, preferably 200 or less. Note that, contrary to this embodiment, the trapping recess 23B can be used as the test liquid recess and the trapping recess 23A can be used as the standard liquid recess.

### Detection Target Image Acquisition Process

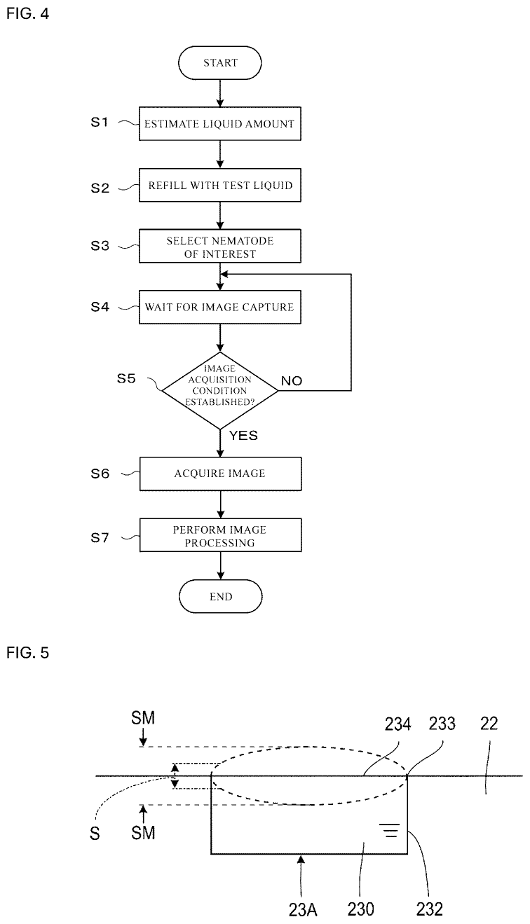

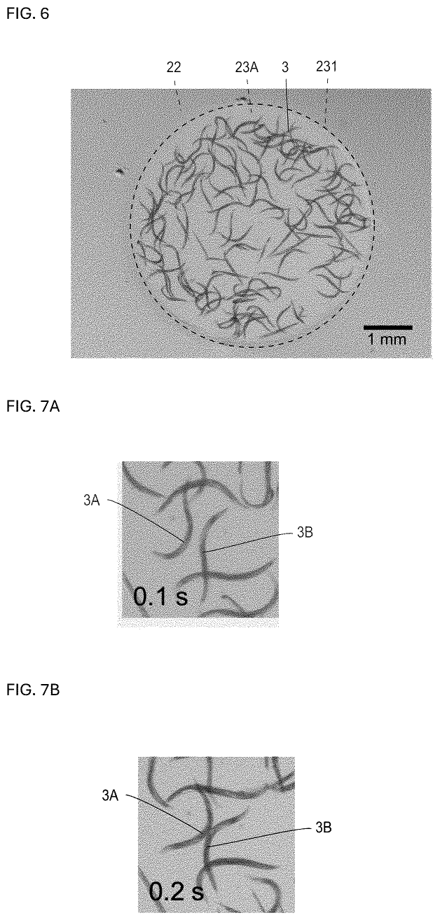

FIG. 4 is a flowchart showing the detection target image acquisition process in which the image recognition device 1 acquires a processed detection target image as the detection target image, FIG. 5 is an enlarged view of the nematode trapping recess 23 A filled with test liquid 230, and FIG. 6 is a diagram showing an example of an unprocessed detection target image acquired by the image recognition device 1.

As shown in FIG. 4, the control unit 11 executes the image acquisition program 125 in response to an external input to the input unit 15, and starts the detection target image acquisition process. At this time, the image acquisition unit 13 is placed directly above the trapping recess 23A (see FIG. 3) to be photographed, and the distance to the trapping recess 23A and the enlargement/reduction are adjusted so that the entire trapping recess 23A fits within the photographing range and the nematodes 3 moving within the trapping recess 23A are not out of focus. Note that the image acquisition process for the trapping recess 23B can be performed in the same procedure as the image acquisition process for the trapping recess 23A.

Before performing photography, the control unit 11 estimates, by using the liquid amount estimation unit 17, the amount of test liquid 230 with which the nematode trapping recess 23A to be photographed is refilled (step S1). More specifically, the control unit 11 confirms the amount of the test liquid 230 in the trapping recess 23A (the height of the liquid level 234 in the trapping recess 23A) using the information obtained from the liquid amount estimation unit 17, and estimates the amount of the test liquid 230 (amount of refill liquid) with which the trapping recess 23A is refilled, in order to bring the amount of the liquid into the state adjusted for image capture, in which the height of the liquid level 234 (see FIG. 5) of the test liquid 230 in the trapping recess 23A after refilling of the test liquid 230 falls within a predetermined range (allowable range S) including the position of the upper edge 233 of the peripheral wall 232 of the trapping recess 23A.

The allowable range S is a range equal to or smaller than half the maximum change range SM defined by the maximum height difference occurring in the liquid level 234 of the test liquid 230, preferably a range equal to or smaller than 30% of the maximum change range SM, and more preferably a range equal to or smaller than 10% of the maximum change range SM. The maximum change range SM is the total range of the upward change range, which is the height width from the height where the liquid level 234 of the test liquid 230 coincides with the upper edge 233 of the peripheral wall 232 of the nematode trapping recess 23A to the height where it becomes maximum due to surface tension, and the downward change range, which is the same height width as the upward change range on the downward side from the height where the liquid level 234 coincides with the upper edge 233.

The amount of the test liquid 230 with which the trapping recess 23A is refilled (amount of refill liquid) may be estimated, for example, by using the liquid amount estimation unit 17 as a timer to calculate the amount of time elapsed since the nematodes were supplied to the vicinity of the central position C of the solid phase 22, or by using the liquid amount estimation unit 17 as a camera disposed diagonally or directly above the trapping recess 23A and determining based on the depiction of the upper edge 233 of the trapping recess 23A. When the liquid amount estimation unit 17 is a camera disposed diagonally, it is sufficient to confirm the amount of the test liquid 230 by photographing with visible light, and when the liquid amount estimation unit 17 is disposed directly above, it measures the distance to the liquid surface as a range finder using infrared rays or the like, and measures how far that distance has dropped from the height at the start of the experiment. When the liquid amount estimation unit 17 is a camera, the image acquisition unit 13 may fulfill the role of the liquid amount estimation unit 17. In addition, when the subject of imaging is the trapping recess 23A which is a test liquid recess and when the subject of imaging is the trapping recess 23B which is a standard liquid recess, the amount of the refill liquid may differ due to differences in the amounts of evaporation of the test liquid and the standard liquid and the amounts of absorption by the solid phase 22. Therefore, even when the trapping recess 23B is the subject of imaging, the amount of the refill liquid is estimated in the same way as for the trapping recess 23A.

The control unit 11 causes the liquid supply unit 18 to refill the trapping recess 23A with the liquid by the amount estimated in step SI so that the height of the liquid level 234 (see FIG. 5) of the test liquid 230 in the nematode trapping recess 23A falls within a predetermined range (allowable range S) including the position of the upper edge 233 of the peripheral wall 232 of the trapping recess 23A (step S2). The liquid supply unit 18 is filled with the test liquid 230 in advance. Note that steps S1 and S2 may be performed by an experimenter who performs the experiment, rather than by the control unit 11. Steps S1 and S2 may also be performed simultaneously. That is, while refilling the test liquid 230 into the trapping recess 23A, the end of refilling may be determined from a change in the depiction of the upper edge 233 of the trapping recess 23A.

The control unit 11 selects a random nematode of interest 3A from the nematodes 3 swimming in the nematode trapping recess 23A, and another random nematode of interest 3B (step S3). At this time, it is preferable that the second nematode of interest 3B is the nematode 3 adjacent to the first nematode of interest 3A. Specifically, it is preferable that the distance between the center of the nematode of interest 3A and the center of the nematode of interest 3B is shorter than the length of the nematode 3 (1 mm in this embodiment).

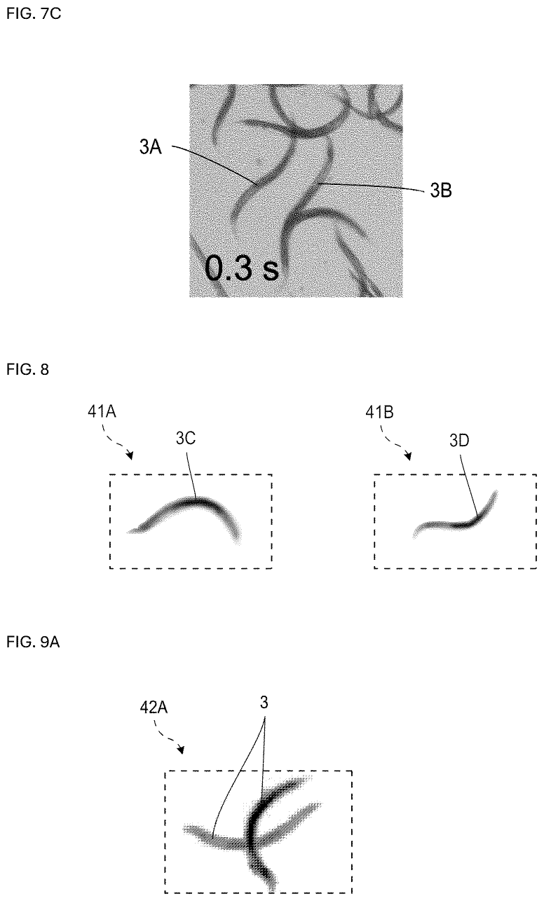

FIG. 7a shows an example of an image of swimming nematodes captured 0.1 seconds (0.1 s) after observation began, FIG. 7b shows an example of an image of swimming nematodes captured 0.2 seconds (0.2 s) after observation began, and FIG. 7c shows an example of an image of swimming nematodes captured 0.3 seconds (0.3 s) after observation began.

Here, the swimming motion of nematodes in liquid will be described in detail. In liquid, nematodes perform swimming motion by constantly repeating flexion and extension at high speed. At this time, one nematode has the property of adjusting the period of movement so as to gradually synchronize the movement with that of its neighboring nematodes. As shown in FIG. 7a, 0.1 seconds (0.1 s) after the start of observation, the nematode of interest 3A is in a flexion state (flexion posture), and the nematode of interest 3B is also in a flexion state. As shown in FIG. 7b, 0.2 seconds (0.2 s) after the start of observation, the nematode of interest 3A is in a further flexion state from that in FIG. 7a, and the nematode of interest 3B is also in a further flexion state from that in FIG. 7a. Then, as shown in FIG. 7c, 0.3 seconds (0.3 s) after the start of observation, the nematode of interest 3A is in an extension state (extension posture), and the nematode of interest 3B is also in an extension state.

The control unit 11 observes the swimming motion of the nematode of interest 3A and the nematode of interest 3B, which repeat flexion and extension, and waits until the image acquisition conditions are met (step S4). The image acquisition conditions include, for example, that the movements of the nematode of interest 3A and the nematode of interest 3B are synchronized, that the distance between one end and the other end of the nematode of interest 3A is at its longest, or that the distance between one end and the other end is 1 mm or more (in an extension state), etc.

The control unit 11 determines whether the image acquisition condition is met (step S5). If the control unit 11 judges that the image acquisition condition is not met (step S5: NO), the process returns to step S4.

If the control unit 11 judges that the image acquisition conditions are met (step S5: YES), it releases the shutter and acquires a photographed image (unprocessed detection target image, i.e., a primary image) (step S6). In the photographed image (unprocessed detection target image) acquired in this manner, as shown in FIG. 6, a large number of nematodes 3 are clearly shown in the trapping recess 23A inside the edge 231.

The control unit 11 performs image processing on the photographed image (unprocessed detection target image) acquired in step S6 (step S7). The contents of the image processing include a process for removing the edge 231 of the nematode trapping recess 23A and a process for making the background shading uniform. This image processing is a photographed image adjustment process for adjusting the photographed image to make it easier to handle. The control unit 11 stores the processed detection target image obtained by performing this image processing on the photographed image as the detection target image in the detection target image database 120. Note that a plurality of photographed images captured at different times for one detection target may be acquired. In this case, the processed detection target image obtained by performing the image processing in step S7 on each image may be used as the detection target image. Also, if the image quality of the photographed image is good, the unprocessed detection target image, which is a photographed image in a state in which no image processing has been performed, may be stored as the detection target image in the detection target image database 120. In the detection target image obtained in this manner (unprocessed detection target image or processed detection target image), as shown in FIG. 6, a large number of nematodes 3 are clearly shown in the trapping recess 23 A inside the edge 231.

Also, for the trapping recess 23B used as the standard liquid recess, the same steps S1 to S7 as for the trapping recess 23A are carried out to estimate the amount of the liquid, refill with the liquid, select the nematode of interest, acquire an image, and perform necessary image processing to create a processed detection target image. In this case, too, an image similar to the unprocessed detection target image shown in FIG. 6 (however, the trapping recess 23A becomes the trapping recess 23B) and the processed detection target image obtained by processing this unprocessed detection target image can be obtained.

### Creating Multiple-Bodies Pattern Images

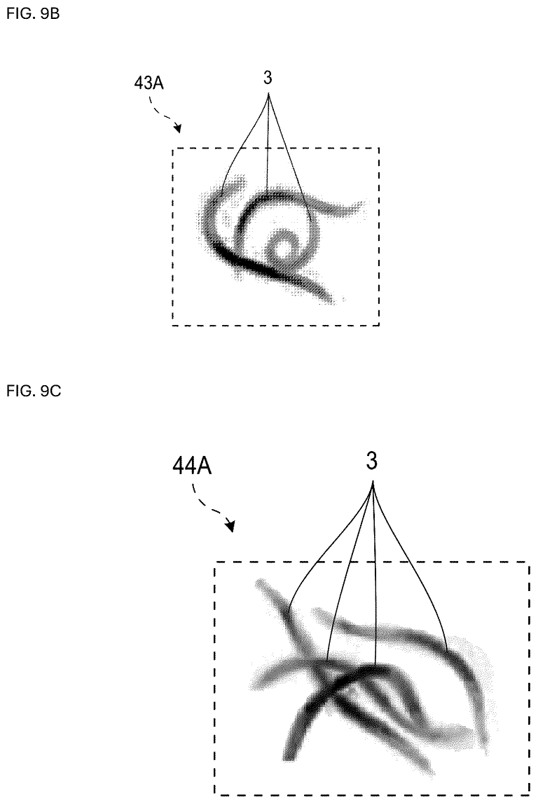

FIG. 8 is a diagram showing examples of two single-body pattern images 41A and 41B respectively showing one nematode 3C and one nematode 3D, FIG. 9a is a diagram showing an example of a label “2” pattern image 42 in which two overlapping nematodes 3 are shown, FIG. 9b is a diagram showing an example of a label “3” pattern image 43 in which three overlapping nematodes are shown, and FIG. 9c is a diagram showing an example of a label “4” pattern image 44 in which four overlapping nematodes are shown.

First, the detection target image (unprocessed detection target image or processed detection target image) acquired in the detection target image acquisition process is selected as a pattern image creation image for creating a single-body pattern image of the nematode and a multiple-bodies pattern image. From this detection target image as the pattern image creation image, the image recognition device 1 creates a single-body pattern image in which one nematode, which is the detection target, is shown. The single-body pattern image can be created, for example, by cutting out non-overlapping nematodes from the detection target image stored in the detection target image database 120 to create a single-body pattern image. In this embodiment, non-overlapping nematodes are cut out from 140 pieces of image data in which 1 to 40 nematodes are shown, and single-body pattern images are created, creating 2,000 single-body pattern images. At this time, for some single-body pattern images, an image by subjecting a single-body pattern image to one or more processes of rotation, enlargement or reduction, and luminance (brightness) change is used as a single-body pattern image different from the original single-body pattern image.

Next, the image recognition device 1 combines the single-body pattern images each showing one nematode that is the detection target to create a multiple-bodies pattern image showing two or more overlapping nematodes. At this time, the image recognition device 1 creates a multiple-bodies pattern image (label N pattern image) with labels corresponding to the number N of nematodes included in the image. The image recognition device 1 preferably creates a label “2” pattern image 42 showing two overlapping nematodes, a label “3” pattern image 43 showing three overlapping nematodes, a label “4” pattern image 44 showing four overlapping nematodes, or a plurality of these, and more preferably creates all of them. In this embodiment, a label “2” pattern image, a label “3” pattern image, and a label “4” pattern image are created by combining a plurality of single-body pattern images. Here, overlapping of a plurality of nematodes means that the images of the nematodes overlap each other partially. It does not matter whether the nematodes in the subject are actually in contact with each other.

In addition, the single-body pattern images to be combined when creating a multiple-bodies pattern image may be the same image or different images. The single-body pattern images are stored in a single-body pattern image database 121, and the multiple-bodies pattern images are stored in a multiple-bodies pattern image database 122.

Here, whether the multiple-bodies pattern image is the label “2” pattern image 42, the label “3” pattern image 43, or the label “4” pattern image 44, it is preferable that the image is an image in which at least a portion of each nematode in the pattern image overlaps other nematodes to be continuous with the other nematodes, and that isolatable nematodes that do not completely overlap other nematodes are not shown. In particular, the label “4” pattern image 44 is also preferable to be an image in which four nematodes are continuous with each other, rather than an image in which there are two images each showing two overlapping nematodes (i.e., an image that can be isolated into two images of two nematodes each). In this way, an image of isolatable nematodes (an image of one nematode for non-overlapping nematodes, and an image of a range in which two or more nematodes overlap somewhere and are continuous with each other in the case of multiple overlapping nematodes) is isolated as a detection target presence area from a detection target image (unprocessed detection target image or processed detection target image) based on a photographed image, and the degree of concordance between the image of this isolated detection target presence area and each pattern image (single-body pattern image and multiple-bodies pattern image) is detected, thereby enabling accurate detection.

It is preferable that the single-body pattern image and the multiple-bodies pattern image of each label are created in equal numbers, and 500 or more of each can be created, preferably 800 or more of each, and more preferably 1,000 or more of each. In this embodiment, 2,000 single-body pattern images and 2,000 pattern images of each label were created.

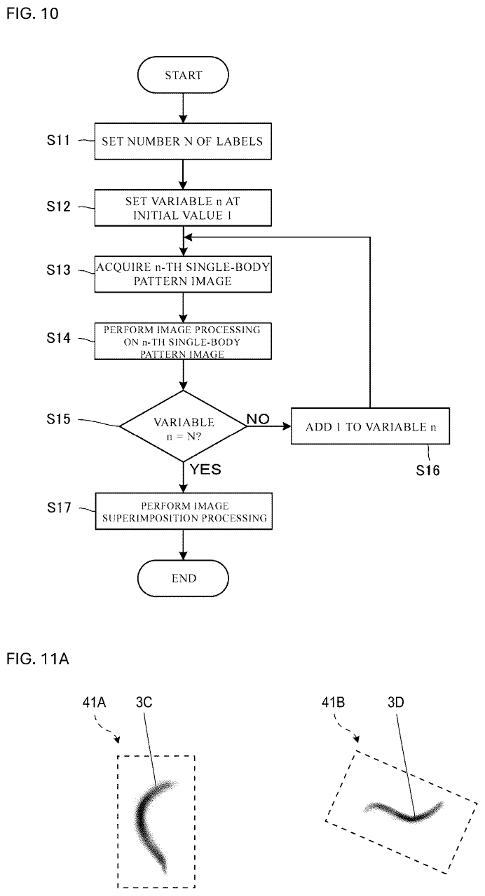

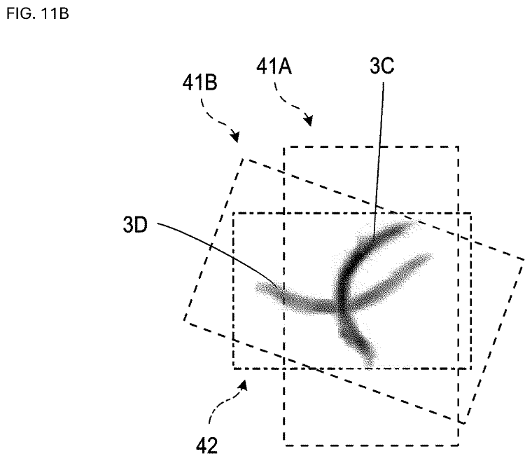

FIG. 10 is a flowchart showing a multiple-bodies pattern image creation process in which the image recognition device 1 creates a multiple-bodies pattern image, FIG. 11a is a diagram showing an example of a processed single-body pattern image, and FIG. 11b is a diagram showing an example of a multiple-bodies pattern image in which processed single-body pattern images are overlaid (superimposed).

The control unit 11 executes the multiple-bodies pattern image creation program 126 in response to an external input to the input unit 15. A method for the image recognition device 1 to create a multiple-bodies pattern image will now be described in detail.

Next, the control unit 11 sets the total number N of single-body pattern images to be combined (the number N of labels of the multiple-bodies pattern image to be created) (step S11), and initializes the variable n (n=1) (step S12). This number N of labels is an integer of 2 or more that indicates how many nematode images are included in the multiple-bodies pattern image. Therefore, when N is 2, n=1 to 2, and a label “2” pattern image 42 is created as the multiple-bodies pattern image. When N is 3, n=1 to 3, and a label “3” pattern image 43 is created. When N is 4, n=1 to 4, and a label “4” pattern image 44 is created as the multiple-bodies pattern image.

Next, the control unit 11 acquires the n-th single-body pattern image (step S13). The acquired pattern image can be a random one from the single-body pattern image data stored in the single-body pattern image database 121.

Next, the control unit 11 performs image processing on the acquired single-body pattern image (step S14). This image processing can be said to be single-body pattern image change processing that changes the single-body pattern image. The contents of the image processing can be a change in luminance, a change in contrast, a change in size (enlargement or reduction), or a combination of any two or more of these changes. Regarding the change in luminance, the change can be within 80% from bright to dark (higher or lower), preferably within 50% for each, and more preferably within 30% for each. Regarding the change in contrast, the change can be within 80% from high to low (higher or lower), preferably within 50% for each, and more preferably within 30% for each. Regarding the change in size, the change can be within 50% from large to small (higher or lower), preferably within 30% for each, and more preferably within 10% for each.

Next, the control unit 11 judges whether image processing of all the single-body pattern images has been completed (step S15). In step S15, the control unit 11 judges whether the variable n is equal to the total number N of the single-body pattern images. If the control unit 11 judges that image processing of all the single-body pattern images has not been completed (step S15: NO), 1 is added to the variable n (step S16), and the process returns to step S13 to acquire a new single-body pattern image. In other words, the processes of steps S13 to S15 are repeated until image processing of all the single-body pattern images to be arranged has been completed.

On the other hand, if the control unit 11 judges that the image processing of all the single-body pattern images has been completed (step S15: YES), image superimposition processing is performed to superimpose each of the image-processed single-body pattern images on top of one another to create a label N pattern image (multiple-bodies pattern image) (step S17). At this time, when the images are superimposed, at least one of the images to be superimposed is subjected to a rotation process (angle change process) for rotating (changing the angle) the image at a random rotation angle, and/or a position change process for randomly changing the position of the image. The rotation process for rotating the image is performed so that the rotation angle is 0 degrees or more and less than 360 degrees. The rotation angle can be freely set as long as it is any one of angles within a range of 0 degrees or more and less than 360 degrees, and may be set randomly. This angle change (rotation process) may be a process of changing the angle to an arbitrary angle in three dimensions (rotating the image in an arbitrary direction), but it is preferable to subject the image to the angle change process (rotation process) on the image plane of the photographed image. This makes it possible to prevent the shape of the nematode from becoming unnatural. In addition, the position change process moves the single-body pattern image in a planar direction (XY direction) within a range in which at least a portion of the single-body pattern image after rotation overlaps at least a portion of another single-body pattern image and all nematodes in the multiple-bodies pattern image being created are continuous with each other.

Here, the superimposition processing of the single-body pattern images will be described in more detail. As a specific example, assume that N is set to 2 and two single-body pattern images 41A and 41B are acquired. In this case, the control unit 11 performs image processing on the single-body pattern images 41A and 41B.

Then, as shown in FIG. 11a, the control unit 11 performs a superimposition processing on the single-body pattern images 41A and 41B. When performing this superimposition processing, the control unit 11 first performs a process of rotating the images. In this embodiment, the single-body pattern image 41A is rotated by 90 degrees, and the single-body pattern image 41B is rotated by 24 degrees. Next, as shown in FIG. 11b, the control unit 11 performs a process of superimposing the single-body pattern image 41A on the single-body pattern image 41B. At this time, the positions of the images are appropriately changed by a position change process, and the images are superimposed so that a part of the nematode 3C shown in the single-body pattern image 41A overlaps a part of the nematode 3D shown in the single-body pattern image 41B.

Thereafter, the control unit 11 stores the created image as a label “2” pattern image 42 in the multiple-bodies pattern image database 122. At this time, the created image is stored in association with numerical data indicating the number (number N) of images of the nematodes (individuals) that are shown in the image and are detection targets. Thus, a label “2” pattern image in which two nematodes that are the detection targets are shown is stored in association with numerical data of N=2, a label “3” pattern image in which three nematodes that are the detection targets are shown is stored in association with numerical data of N=3, and a label “4” pattern image in which four nematodes that are the detection targets are shown is stored in association with numerical data of N=4. Specifically, the processes in steps S11 to S17 are repeated as many times as the required number of patterns for each required number of N (number of images of the nematodes that are the detection targets) to create the required multiple-bodies pattern images. Here, the single-body pattern image randomly acquired in step S13 may be the same single-body pattern image acquired in the repeated steps S11 to S17. Even in such a case, the completed multiple-bodies pattern image will be different because the luminance, contrast, size, and angle of the single-body pattern image are changed by the image processing and superimposition processing. Also, the same single-body pattern image may be acquired in each step S13 in one execution of steps S11 to S17, and in this case also, the completed multiple-bodies pattern image will be different because the luminance, contrast, size, and angle of the single-body pattern image are changed.

In this embodiment, the image processing (step S12) was performed on the acquired single-body pattern images before the process of superimposing each of the single-body pattern images on top of one another (step S16), but the image processing of step S14 may be performed after the superimposition processing of step S16. Also, although the luminance change, contrast change, and size change in the image processing are separated from the angle change process in the superimposition processing, any appropriate configuration may be used, such as performing luminance change, contrast change, size change, angle change, or one or more of these in the image processing, or eliminating the prior image processing and performing luminance change, contrast change, size change, angle change, or one or more of these on each single-body pattern during the superimposition processing.

### Model Image Creation

The control unit 11 creates a model image for learning by combining the single-body pattern image stored in the single-body pattern image database 121, the multiple-bodies pattern image stored in the multiple-bodies pattern image database 122, and a noise image stored in the noise image database 123.

The noise image is an image that does not show the detection target, but shows noise that may be erroneously recognized (misidentified) as the detection target in image recognition. Such noise is, for example, the edge of a liquid recess, or dust or microorganisms contained in the liquid. Each noise image may be tagged with a tag indicating the type of noise it is. For example, a noise image showing the edge of a liquid recess may be tagged with the edge of the liquid recess. Furthermore, numerical data N=0 is associated with the noise image data.

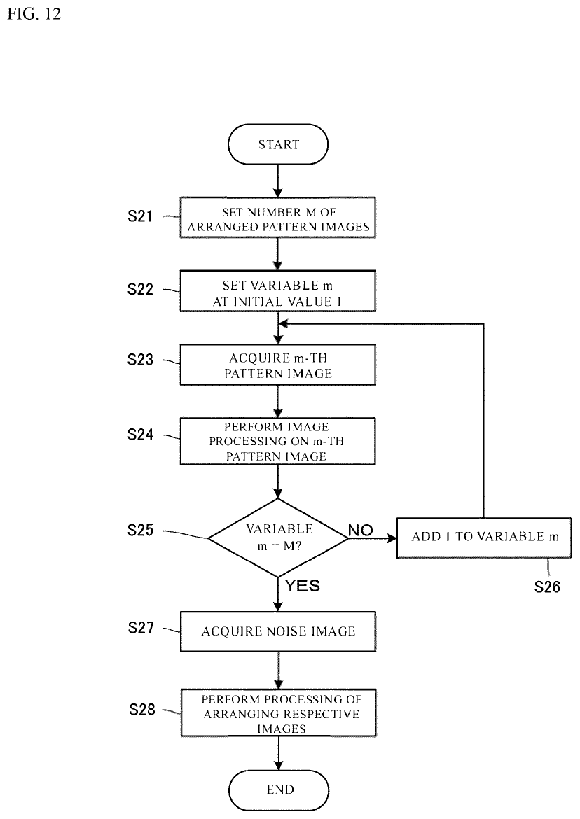

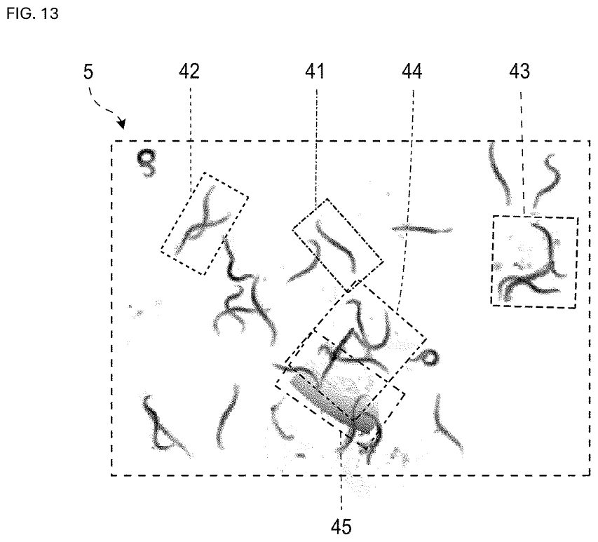

FIG. 12 is a flowchart showing a model image creation process in which the image recognition device 1 creates a model image for learning, and FIG. 13 is a diagram showing an example of a model image 5 created by the image recognition device 1.

In this embodiment, the model image 5 was created by combining multiple single-body pattern images, label “2” pattern images, label “3” pattern images, label “4” pattern images, and noise images.

The control unit 11 executes the model image creation program 127 in response to an external input to the input unit 15.

Next, the control unit 11 sets the total number M of arranged pattern images (step S21), and initializes the variable m (m=1) (step S22).

Next, the control unit 11 acquires the m-th pattern image (step S23). The acquired pattern image can be a random one from among the single-body pattern image data stored in the nematode single-body pattern image database 121 and the multiple-bodies pattern image data stored in the multiple-bodies pattern image database 122.

Next, the control unit 11 performs image processing on the acquired pattern image (step S24). The contents of image processing can be, for example, one or more of the following processes: changing the luminance (brightness) within 30% higher or lower, changing the contrast within 30% higher or lower, and enlarging or reducing within 10%. When performing the process of changing the luminance (brightness), it is preferable to perform a process to lower the luminance (brightness) the larger the value of N of the numerical data.

Next, the control unit 11 judges whether the image processing of all pattern images has been completed (step S25). In step S25, the control unit 11 judges whether the variable m is equal to the total number M of arranged pattern images. If the control unit 11 judges that image processing of all pattern images has not been completed (step S25: NO), 1 is added to the variable m (step S26), and the process returns to step S23 to acquire a new pattern image. In other words, the processes of steps S23 to S25 are repeated until image processing of all pattern images to be arranged is completed. At this time, it is preferable that the end condition of the image processing is when at least the number of pattern images acquired so far is one or more for each of the nematode single-body pattern image, label “2” pattern image (image associated with numerical data of N=2), label “3” pattern image (image associated with numerical data of N=3), and label “4” pattern image (image associated with numerical data of N=4).

On the other hand, if the control unit 11 judges that image processing of all pattern images has been completed (step S25: YES), the noise image to be arranged is acquired (step S27). In step S26, one or more random noise images 45 are extracted from the noise image database 123. At this time, it is preferable to extract at least one noise image 45 with a different tag attached thereto.

Then, the control unit 11 randomly arranges the image-processed pattern images (single-body pattern image 41, label “2” pattern image 42, label “3” pattern image 43, label “4” pattern image 44) and the noise image 45 to create the model image 5 (step S28). When arranging the images, each image may be rotated at a random rotation angle.

After that, the control unit 11 stores the created model image 5 in the model image database 124 and ends the creation of the model image 5. At this time, data of each pattern image included in the created model image 5, data indicating the position (correct position) of each pattern image on the model image 5, and data of the number of nematodes included in the model image 5 (number of correct answers) calculated from each label pattern image and single-body pattern image used are stored as model image status data in association with the model image 5. Note that, in this embodiment, the image processing (step S24) of each pattern image is performed before the process of arranging the single-body pattern image and the multiple pattern images (step S28), but the image processing of step S24 may be performed after the arrangement process of step S28. Also, the number of model images to be created can be 1,000 or more, preferably 10,000 or more, and more preferably 20,000 or more. In this embodiment, 20,000 model images were created.

### Image Recognition Learning Program

The learning program 128 stored in the storage unit 12 is a program for detecting a detection target, and is a deep learning program using artificial intelligence (AI). Deep learning makes it possible to learn in the expression of very complex features by making the intermediate layer (the part that performs calculations between input data and output data) of a neural network multi-layered (deep neural network) and learning. In detail, it is a learning method that creates a network that combines various calculations (layers) including convolution integrals with various parameters, and extracts features from an image. In this embodiment, deep learning of YOLOv4 (You Only Look Once) (Bochkovskiy A, YOLOv4: Optimal Speed and Accuracy of Object Detection, arXiv: 2004.10934v1) was used. Note that other programs than YOLOv4 may be used for deep learning. Also, a GPU computing unit was used for deep learning.

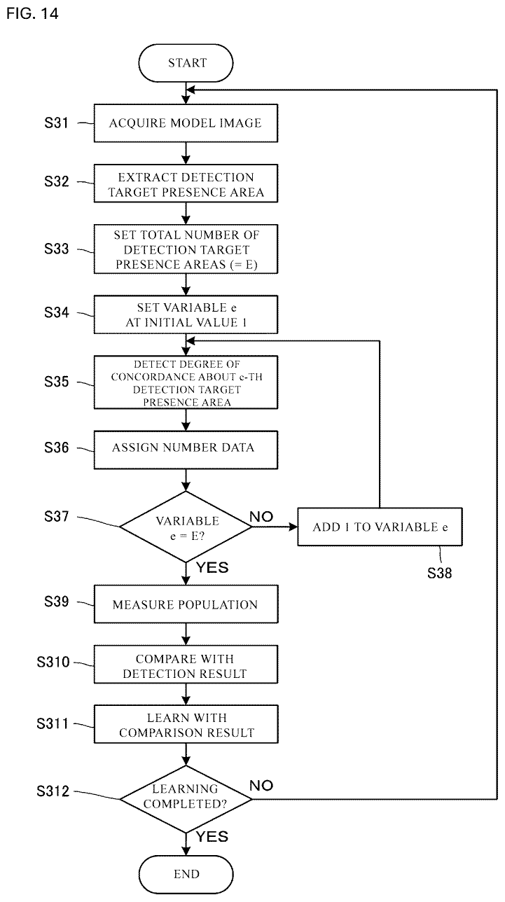

FIG. 14 is a flowchart showing a learning process in which the image recognition device 1 learns using a model image.

The control unit 11 executes the learning program 128 in response to an external input to the input unit 15.

The control unit 11 randomly extracts one of the model images stored in the model image database 124 (step S31).

Next, the control unit 11 extracts detection target presence areas from the model image that possibly includes an image of a nematode (step S32), sets the total number E of extracted detection target presence areas (step S33), and initializes a variable e (e=1) (step S34).

Next, the control unit 11 detects the degree of concordance between the e-th detection target presence area and each pattern image (single-body pattern image and multiple-bodies pattern image) (step S35). If the multiple-bodies pattern images of nematodes are three types, a label “2” pattern image, a label “3” pattern image, and a label “4” pattern image, the control unit 11 detects the degree of concordance between each of the four types of pattern images including the single-body pattern image×the number of patterns (if there are 2,000 of each, ×2,000).

The control unit 11 assigns the numerical data associated with the pattern image showing the highest degree of concordance to the e-th detection target presence area as data on the number of nematodes (step S36). In this embodiment, the numerical data includes five types of data: data showing the number of nematodes “0” (not matching any pattern image or equivalent to a noise image), data showing the number of nematodes “1” (matching the single-body pattern image), data showing the number of nematodes “2” (matching the label “2” pattern image), data showing the number of nematodes “3” (matching the label “3” pattern image), and data showing the number of nematodes “4” (matching the label “4” pattern image).

Next, the control unit 11 judges whether or not the setting of numerical data to all detection target presence areas has been completed (step S37). In step S37, the control unit 11 judges whether the variable e is equal to the total number E of detection target presence areas. Note that step S37 may be configured to determine whether e is less than E, assuming that e<E. If the control unit 11 judges that the assignment of numerical data to all detection target presence areas has not been completed (step S37: NO), 1 is added to the variable e (step S38), and the process returns to step S35, where the degree of concordance with each pattern image is detected for the detection target presence areas to which the assignment of numerical data has not been completed. In other words, the processes of steps S35 to S38 are repeated until the assignment of numerical data to all detection target presence areas has been completed.

On the other hand, if the control unit 11 judges that the assignment of numerical data to all detection target presence areas has been completed (step S37: YES), the control unit 11 counts the population in the model image by adding up all the numerical data assigned to each detection target presence area in step S36 (i.e., the population in the detection target presence areas) (step S39).

Then, the control unit 11 compares the model image status data associated with the model image with the image recognition result (step S310). The contents to be compared can be contents corresponding to both the determination result by image recognition and the model image status data, such as the determined pattern image result and the actual arrangement of the pattern image, the determined number of nematodes and the actual number of nematodes, and the result determined as noise and the actual arrangement of the noise image.

The control unit 11 reflects (feeds back) the comparison results of the determined contents (step S311). Therefore, by repeating the feedback, the number of arrangement patterns of single-body pattern images and multiple-bodies pattern images that can be accurately determined is increased and learned. In addition, in order to learn about the arrangement patterns of noise images, the number of non-nematode patterns that can be accurately determined is increased and learned.

The control unit 11 judges whether all learning is complete (step S312). The completion of learning can be judged by criteria such as when a preset number of learning times is reached, when learning is completed for all model images, or when the accuracy of the determination results reaches a specific percentage or higher. If the control unit 11 judges that all learning is complete (step S312: YES), learning ends. If the control unit 11 judges that all learning is not complete (step S312: NO), the process returns to step S31 and learning is performed on a new model image. In other words, the processes of steps S31 to S312 are repeated until learning is complete.

### Counting the Population of Detection Targets

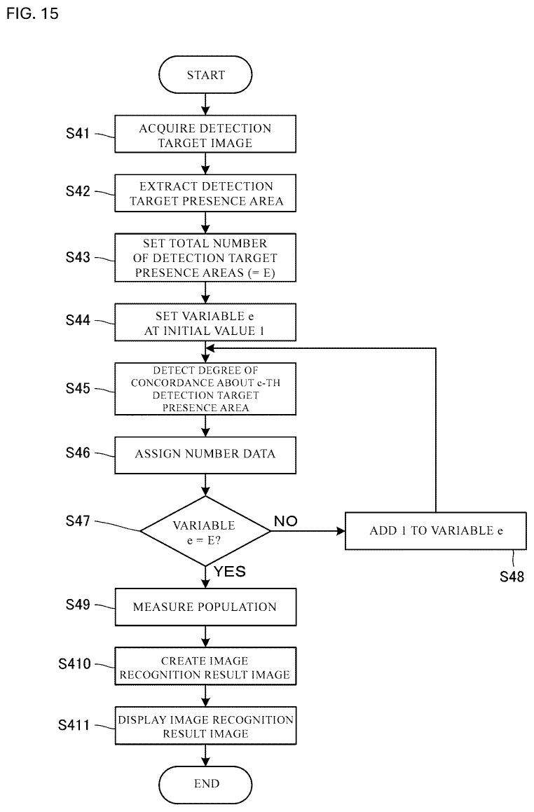

FIG. 15 is a flowchart showing a detection target population counting process in which the image recognition device 1 acquires a detection target image (an unprocessed detection target image or a processed detection target image) and performs counting processing on the population of detection targets.

The control unit 11 executes the counting program 129 in response to an external input to the input unit 15.

When the control unit 11 starts the population counting process in response to an external input to the input unit 15, the control unit 11 first acquires a specific detection target image from the detection target image database 120 (step S41). That is, the control unit 11 acquires the detection target image (unprocessed detection target image or processed detection target image) selected by the user of the image recognition device 1 as the image in which the population of nematodes is to be counted.

Next, the control unit 11 extracts detection target presence areas that possibly includes an image of a nematode from the detection target image (step S42), sets the total number E of extracted detection target presence areas (step S43), and initializes a variable e (e=1) (step S44).

Next, the control unit 11 detects the degree of concordance between the e-th detection target presence area and each pattern image (nematode single-body pattern image and nematode multiple-bodies pattern image) (step S45). If the multiple-bodies pattern images are of three types, a label “2” pattern image, a label “3” pattern image, and a label “4” pattern image, the control unit 11 detects the degree of concordance between each of the four types of pattern images including the single-body pattern image×the number of patterns (if there are 2,000 of each, ×2,000).

The control unit 11 assigns the numerical data associated with the pattern image showing the highest degree of concordance to the e-th detection target presence area as data on the number of nematodes (step S46). In this embodiment, the numerical data includes five types of data: data showing the number of nematodes “0” (not matching any pattern image or equivalent to a noise image), data showing the number of nematodes “1” (matching the single-body pattern image), data showing the number of nematodes “2” (matching the label “2” pattern image), data showing the number of nematodes “3” (matching the label “3” pattern image), and data showing the number of nematodes “4” (matching the label “4” pattern image).

Next, the control unit 11 judges whether or not the setting of numerical data to all detection target presence areas has been completed (step S47). In step S47, the control unit 11 judges whether the variable e is equal to the total number E of detection target presence areas. Note that step S47 may be configured to determine whether e is less than E, with e<E. If the control unit 11 judges that the assignment of numerical data to all detection target presence areas has not been completed (step S47: NO), 1 is added to the variable e (step S48), and the process returns to step S45, where the degree of concordance with each pattern image is detected for the detection target presence areas to which the assignment of numerical data has not been completed. In other words, the processes of steps S45 to S48 are repeated until the assignment of numerical data to all detection target presence areas has been completed.

On the other hand, if the control unit 11 judges that the assignment of numerical data to all detection target presence areas has been completed (step S47: YES), the control unit 11 counts the population in the detection target image by adding up all the numerical data assigned to each detection target presence area in step S46 (i.e., the population in the detection target presence areas) (step S49).

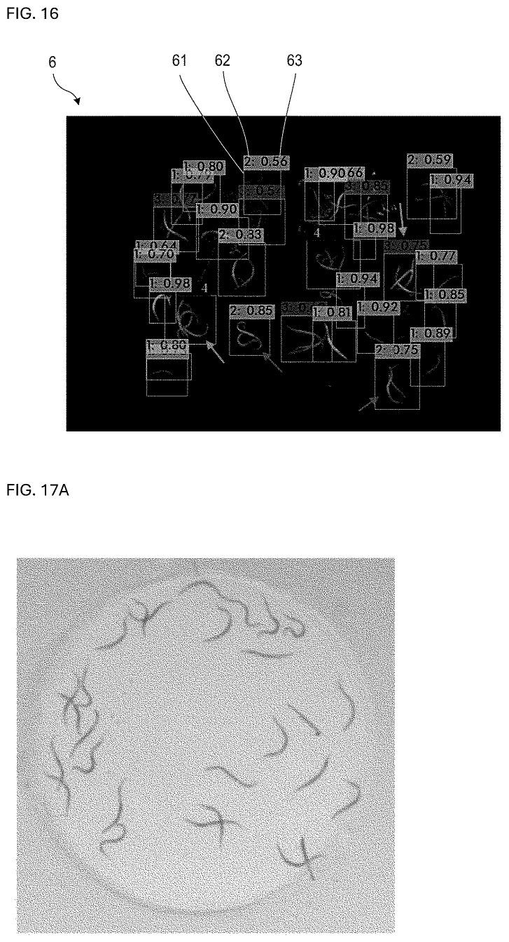

Then, an image recognition result image 6 (see FIG. 16 described below) showing the image recognition result of the detection target is created (step S410), the image recognition result image 6 is displayed on the display unit 14 (step S411), and the population counting process is terminated.

Furthermore, if multiple detection target images of one detection target captured at different times are stored, the population can be counted more accurately. In detail, the number of individuals can be calculated for all detection target images and the average value calculated from the calculation results can be used as the calculation result, or the top 95% of the population count results can be used as the calculation result excluding outliers such as the maximum and minimum. With this method, the population can be counted more accurately.

FIG. 16 is a diagram showing an example of the image recognition result image 6 showing the image recognition result of the detection target.

The image recognition result image 6 displays, as display items, a pattern image area 61 contained in the detection target image determined by the image recognition device 1, the number of nematodes 62 contained in the pattern image area 61, and a result accuracy index 63 indicating the accuracy of the detection result. The population of all nematodes contained in the detection target image (the population calculated in step S49) is the population count result, and is displayed in a separate image (not shown).

The pattern image area 61 indicates the area (detection target presence area) corresponding to the pattern image determined by the control unit 11 for the nematodes contained in the detection target image. For example, when two nematodes overlap, the area including these nematodes is recognized as a label “2” pattern image and this area is displayed. Note that areas determined to be noise images are not displayed as pattern image area 61.

The number of nematodes 62 indicates the nematode population data associated with the pattern image determined by the control unit 11 in the pattern image area 61. For example, when two nematodes overlap, the area including these nematodes is recognized as a label “2” pattern image and is displayed as 2.

The result accuracy index 63 indicates the degree of concordance between the arrangement of the recognized nematodes and the pattern image determined by the control unit 11. The degree of concordance indicates the degree to which the range of the recognized nematodes matches the range of the nematodes in the determined pattern image, and is calculated, for example, based on the number of pixels. When the number of pixels is used as the basis, the degree of concordance is calculated by dividing (the number of pixels in the range of the nematodes contained in the determined pattern image) by (the number of pixels of overlapping nematodes at the position where the nematodes contained in the pattern image and the recognized nematode overlap most), with the maximum value calculated as 1. Note that the degree of concordance is not limited to this and can be calculated by any known appropriate method.

With the above configuration, it is possible to provide an image recognition program capable of accurately counting the population of detection targets even when a plurality of detection targets overlap each other, an image recognition device using the same, a detection target population counting method, and a model image creation device for image recognition learning to be used therefor.

In the image recognition device 1, the control unit 11 acquires a detection target image (unprocessed detection target image or processed detection target image) showing that nematodes that are a plurality of detection targets and stores in the storage unit 12 a single-body pattern image showing one detection target and a multiple-bodies pattern image showing the plurality of detection targets, and the control unit 11 detects the degree of concordance with each pattern image to distinguish the nematodes, which are the plurality of detection targets, included in the detection target image. With this configuration, it is possible to more accurately distinguish and count the population of nematodes, taking into account the overlap of nematodes, than using only a single-body pattern image showing one nematode.

Furthermore, the multiple-bodies pattern image of the nematode is created by combining one or more single-body pattern images selected from the multiple single-body pattern images. This configuration eliminates the need to prepare images of multiple nematodes as multiple-bodies pattern images by taking a huge number of photographs, which would normally be required, improving convenience. Furthermore, multiple-bodies pattern images can be created as many times as there are combinations of single-body pattern images, improving the randomness of the model image. Therefore, when the image recognition device 1 uses a model image for learning, more efficient learning can be achieved.

Furthermore, the multiple-bodies pattern image of the nematode is created by overlapping one or more single-body pattern images selected from the plurality of single-body pattern images, and by subjecting one or more single-body pattern images among the one or more single-body pattern images selected from the plurality of single-body pattern images to a change in at least one of a position in a planar direction or a rotation angle on a plane. With this configuration, it is possible to create more multiple-bodies pattern images in which nematodes are overlapped in different ways. Furthermore, even with the same combination of single-body pattern images, many multiple-bodies pattern images can be created by changing the position, rotation angle, and luminance (brightness) of the single-body pattern images.

Furthermore, the detection target image is either an unprocessed detection target image captured in a state adjusted for image capture in which the liquid level 234 of the test liquid 230 in the nematode trapping recess 23A (liquid recess) is in a range equal to or smaller than half the maximum change range SM defined by the maximum height difference caused by surface tension on the liquid level 234, or a processed detection target image obtained by performing appropriate image processing such as processing to remove unnecessary parts such as edges and processing to make the background shading uniform on this unprocessed detection target image. With this configuration, almost no shadows due to the lens effect are generated in the photographed unprocessed detection target image and the processed detection target image obtained by subjecting the unprocessed detection target image to image processing, improving the detection accuracy of nematodes.

Furthermore, when acquiring a photographed image to be used as an unprocessed detection target image, the control unit 11 executes step S1 in which the liquid amount estimation unit 17 estimates the amount of test liquid 230 to be refilled into the nematode trapping recess 23A (liquid recess) to be photographed, and step S2 in which the liquid supply unit 18 refills the trapping recess 23A with the liquid by the amount estimated in step S1. This configuration makes it possible to make the liquid level 234 as close to a flat surface as possible, and prevents diffuse reflection of light, image distortion, and reflection of shadows around the trapping recess 23A (for example, the edge 231 of the trapping recess 23A, the peripheral wall 232 of the trapping recess 23A, the upper edge 233 of the trapping recess 23A, or a combination of these) caused by distortion (concave or convex) of the liquid level 234 of the test liquid 230, and makes it possible to acquire a clear image.

Furthermore, 0 to 100 nematodes are present per 0.01 mm² of an opening area of each of the nematode trapping recesses 23A and 23B (liquid recess) when viewed from the upper side of the nematode trapping recesses 23A and 23B (liquid recess). This configuration ensures the accuracy of assay results using nematodes while preventing the nematodes from overlapping too much in the liquid recess.

The nematode trapping recesses 23A and 23B (liquid recess) have a depth T of 1 to 5 mm and a diameter R of 5 mm. The trapping recesses 23A and 23B are adjusted so that 0 to 200 nematodes can swim in the trapping recesses 23A and 23B, while the diameter R is 5 mm. This configuration ensures the accuracy of assay results using nematodes while preventing the nematodes from overlapping too much in the liquid recess. It is also possible to ensure a liquid amount that will not cause all of the liquid (test liquid or standard liquid) to evaporate or be absorbed into the solid medium until the nematodes move to the trapping recess 23. Furthermore, the background of the detection target image (unprocessed detection target image or processed detection target image) is a moderate color that is neither too dark nor too bright, improving the detection accuracy of the nematodes. Furthermore, when there are two or more swimming nematodes, the population of nematodes can be counted accurately whether they are separated or overlapping in the photographed unprocessed detection target image and the processed detection target image obtained by performing appropriate image processing on the unprocessed detection target image, and when there are 10 or more nematodes, they can be counted with higher detection accuracy than conventional detection methods.

Furthermore, the control unit 11 focuses on two or more nematodes, photographs the nematodes of interest in an extension state, and acquires an unprocessed detection target image (primary image), and either uses, as the detection target image, this unprocessed detection target image as is, or uses, as the detection target image, a processed detection target image (secondary image) obtained by performing appropriate image processing on this unprocessed detection target image. With this configuration, it is possible to acquire a detection target image (unprocessed detection target image or processed detection target image) with most of the nematodes being in an extension state, by utilizing the property of nematodes to synchronize their movements with other adjacent nematodes. Therefore, it is possible to significantly reduce the detection of nematodes in a flexion state where they overlap in a complex manner, and it is possible to perform image recognition on nematodes in an extension state where individuals barely overlap each other, thereby improving the accuracy of nematode detection.

Furthermore, the control unit 11 creates a model image by compositing (combining) a single-body pattern image showing one of the detection targets, a multiple-bodies pattern image corresponding to an image showing two or more of the detection targets overlapping each other, and a noise image showing none of the detection targets and including noise. With this configuration, the accuracy of determining each pattern image can be improved by using the created model image for image recognition training. Also, noise such as the edge of the liquid recess will no longer be mistakenly determined to be nematodes, improving the accuracy of counting the nematode population.

Furthermore, each pattern image used in the model image is used with its luminance (brightness) and contrast changed. This configuration allows the image recognition device 1 to learn about differences in luminance (brightness) and contrast, improving the accuracy of counting the nematode population. In particular, because the luminance (brightness) and contrast are changed for each pattern image, it is possible to mix luminance (brightness) and contrast that can change depending on whether the nematodes are swimming in a shallow area near the surface of the liquid recess or in a deep area near the bottom, and it is possible to create a variety of pattern images that are close to actual photographed images, improving the accuracy of counting the nematode population.

In addition, the control unit 11 performs image processing for removing the edges of the liquid recess and for making the shading of the background uniform for the acquired unprocessed detection target image (primary image) to create a processed detection target image, and uses the processed detection target image as a detection target image. This configuration allows the nematodes shown in the processed detection target image as the detection target image to be displayed more clearly, thereby improving the detection accuracy of the nematode population.

In addition, the control unit 11 acquires a plurality of unprocessed detection target images captured at different times for one detection target, performs appropriate image processing on the unprocessed detection target images to obtain a processed detection target image, and sets the processed detection target image as the detection target image. With this configuration, a calculation method for nematodes using a plurality of different detection target images can be used. More specifically, the calculation method can be a method of calculating the number of nematodes for all detection target images, calculating an average value from the calculation results, or calculating the top 95% from the population count results excluding outliers such as the maximum and minimum.

Furthermore, the same number of single-body pattern images and multiple-bodies pattern images are created. With this configuration, each pattern image is used equally for determination during learning, and there is no difference in the amount of learning for each pattern image. Therefore, when the image recognition device 1 performs image recognition of the detection target image, it does not recognize any one pattern image preferentially, and the detection accuracy of nematodes can be improved.

The present invention is not limited to the above-described embodiments and can take various forms.

For example, in this embodiment, the focus was on two nematodes swimming in real time and the two nematodes were photographed when they were in an extension state to acquire an unprocessed detection target image, but it is also possible to photograph a video of nematodes swimming in a liquid recess, focus on the two nematodes swimming in the video, and acquire an unprocessed detection target image by photographing the two nematodes when they are in an extension state.

Furthermore, in this embodiment, the detection target is a nematode, but the detection target can be something other than a nematode. In particular, the image recognition device 1 of the present invention can be suitably used in cases where the detection target has an elongated shape and multiple detection targets overlap within one area. Examples of such detection targets include parasites, earthworms, and eels.

In addition, various programs of the present invention, such as the population counting program 129 (image recognition program), learning program 128, model image creation program 127, multiple-bodies pattern image creation program 126 and image acquisition program 125, can be stored in a non-temporary (non-transient) tangible storage medium and provided as a program storage medium 19.

The detection target image acquisition means and detection target image acquisition unit of the present invention correspond to the control unit 11 which executes the image acquisition program 125 described in the embodiment, and similarly below, the extraction means corresponds to the control unit 11 which executes the step of extracting the detection target presence area (step S42), the storage means corresponds to the storage unit 12, the image recognition means corresponds to the control unit 11 which executes the step of counting the population of nematodes in the detection target image (step S49), the image recognition program corresponds to the population counting program 129 which includes the step of counting the population of nematodes in the detection target image (step S49), the multiple-bodies pattern image creation means corresponds to the control unit 11 which executes the multiple-bodies pattern image creation program 126, the noise image acquisition unit corresponds to the control unit 11 which executes the step of acquiring a noise image (step S27), the model image creation unit corresponds to the control unit 11 that executes the model image creation program 127, the label multiple-bodies pattern image corresponds to the label “3” pattern image and the label “4” pattern image, the single-body pattern image acquisition unit and the multiple-bodies pattern image acquisition unit correspond to the control unit 11 that executes the step of acquiring each pattern image in the creation of the model image (step S23), the extraction unit corresponds to the control unit 11 that executes the step of extracting the detection target presence area (step S42), the recognition unit corresponds to the control unit 11 that executes the step of detecting the degree of concordance of the detection target presence area (step S45), the liquid amount estimation means corresponds to the control unit 11 and the liquid amount estimation unit 17 that execute step S1, and the liquid supply means corresponds to the control unit 11 and the liquid supply unit 18 that execute step S2, but this invention is not limited to this embodiment and can be various other embodiments. Also, the screens and specific configurations etc. listed in the above-mentioned embodiment are only examples and can be appropriately changed according to the actual product.

For example, in the above embodiment, the single-body pattern image and the detection target image are acquired by photographing them with the image acquisition unit 13, but the single-body pattern image and the detection target image may be images input from an external device other than the image recognition device 1. In this case, the image recognition device 1 acquires the images to be the single-body pattern image and the detection target image from the external device via the communication unit 16 or a storage medium such as a USB memory.

In the above-mentioned embodiment, the detection target image is an unprocessed detection target image with good shooting conditions and clear image quality, or a processed detection target image obtained by subjecting the unprocessed detection target image to appropriate image processing such as processing to remove unnecessary parts such as edges and processing to make the background shading uniform, and this detection target image is used as a pattern image creation image. From this pattern image creation image, a single-body pattern image showing one nematode as the detection target is created, and this is processed and composited to create a multiple-bodies pattern image showing multiple nematodes. Not limited to this, a single-body pattern image of a nematode may be created from detection target images acquired under various shooting conditions, and this may be processed and composited to create a multiple-bodies pattern image. In this case, even if various images captured under different shooting conditions are used as the detection target images, it is possible to obtain a good population count result. A specific explanation will be given below.

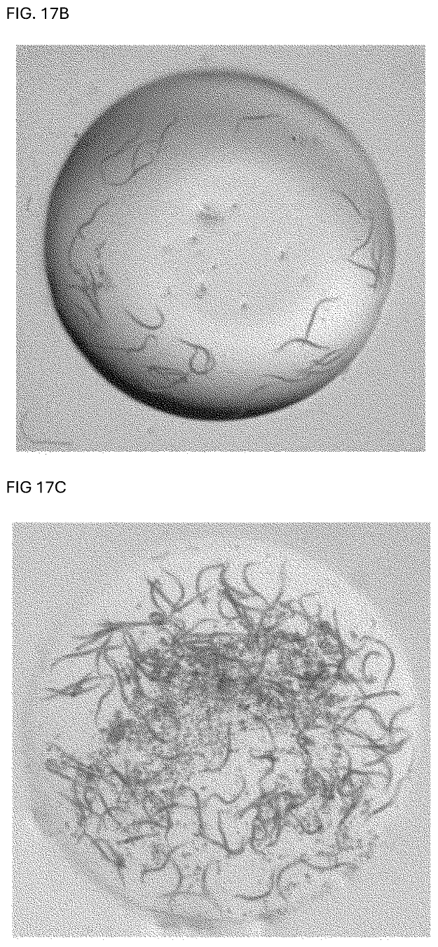

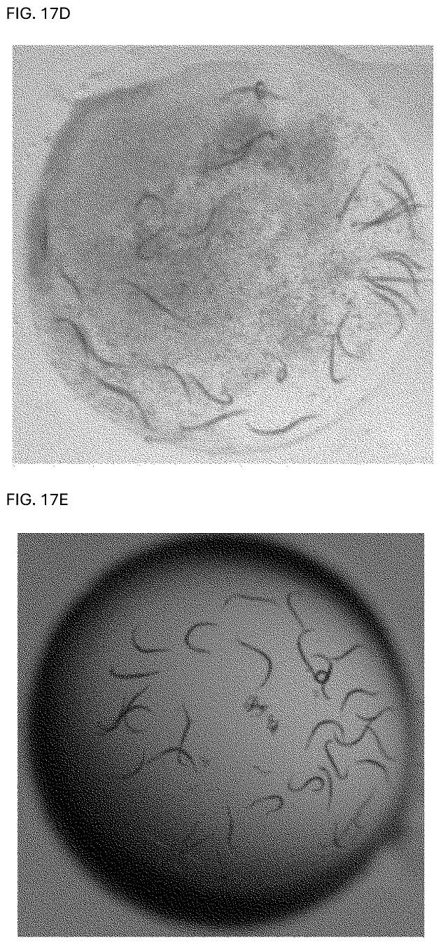

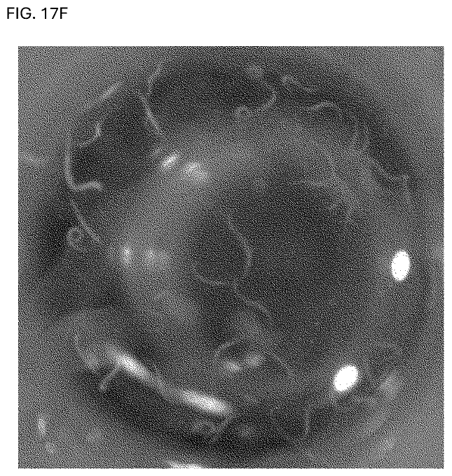

FIGS. 17a to 17f are unprocessed detection target images captured from above with a digital camera mounted on a stereo microscope with a transmitted light illumination device, of a nematode swimming in liquid (inside the liquid recess) injected into a nematode trapping recess with a diameter of 5 mm on a nematode trap plate.

FIG. 17a shows an example of an unprocessed detection target image captured with a suitable amount of liquid and under suitable lighting, similar to the embodiment described above, in which the nematodes in the liquid that are the detection targets are firmly in focus and there are almost no shadows or reflections.

FIG. 17b shows an example of an unprocessed detection target image in which only a small portion of the nematodes in the liquid is in focus.

FIG. 17c shows an example of an unprocessed detection target image in which nematodes in a liquid (test liquid) containing a biological sample mixed with impurities are captured. The contaminants in this image are mainly solid components of the biological sample.

FIG. 17d shows an example of an unprocessed detection target image in which nematodes in a turbid liquid (test liquid) containing a biological sample are captured.

FIG. 17e shows an example of an unprocessed detection target image captured with strong shadows on the edge of the liquid recess.

FIG. 17f shows an example of an unprocessed detection target image that shows a lot of reflected light from the surface of the liquid recess.

When creating single-body pattern images, multiple-bodies pattern images, and detection target images from these unprocessed detection target images, the image recognition device 1 may assign a shooting type to each image with different shooting conditions (image quality). The learning program 128 may then be configured to create multiple model images 5 for each shooting type and learn for each shooting type. This makes it possible to categorize the detection target images by shooting type, recognize shooting types that are the same as or similar to the shooting conditions of the detection target images for which it is desired to count the population of detection targets, and count the population of detection targets using the single-body pattern images and multiple-bodies pattern images created from the detection target images of this shooting type, thereby improving the accuracy of counting the population.

Furthermore, the learning program 128 may create multiple model images 5 by mixing single-body pattern images and multiple-bodies pattern images of different shooting types, and learn using the model images 5 with a mixture of shooting types. In this way, when it is unknown under what shooting conditions the unprocessed detection target images that are the basis of the detection target images were captured, detection can be performed using a mixture of single-body pattern images and multiple-bodies pattern images created from detection target images of multiple shooting types. In this case, it is possible to improve the accuracy of counting the population in the detection target images based on the unprocessed detection target images acquired under various shooting conditions.

Furthermore, the image recognition device 1 may acquire noise images from the acquired photographed images of various image qualities, and store them in the noise image database 123. In this case, the image recognition device 1 acquires nematode images and noise images from photographed images having different image quality factors, such as focus, the type (size, shape, color, etc.) and amount of solid components (impurities) and particles contained in the liquid to be photographed, the turbidity of the test liquid (typically, how high the absorbance is at the wavelength of the imaging light source, and if limited to visible light, how high the turbidity is), the intensity of shadows, or the amount of reflection, creates a single-body pattern image and a multiple-bodies pattern image using the acquired nematode image, and randomly arranges the created single-body pattern image, the created multiple pattern image, and the acquired noise image. This allows multiple model images 5 with image quality different from that of the embodiment to be created, and the image recognition device 1 can learn using model images 5 that mimic various image qualities. That is, in the above-mentioned embodiment, not only in the detection target image shown in FIG. 17a, in which the nematodes in the liquid that are the detection targets are firmly in focus and there are almost no shadows from the edge of the liquid recess or reflections from the liquid level, but also in detection target images of various image qualities, the population of nematodes that are the detection targets can be correctly recognized.

Furthermore, the image recognition device 1 may acquire images of nematodes from a plurality of photographed images with different image quality, and use each image of the nematode as a single-body pattern image to create one multiple-bodies pattern image. That is, images of nematodes may be acquired from two photographed images with different image quality, and a single-body pattern image of the nematode may be created for each of them as in the embodiment and stored in the single-body pattern image database 121, and a multiple-bodies pattern image may be created by combining the single-body pattern images created from the two photographed images with different image quality. With this configuration, even if the detection target image contains a mixture of focused and unfocused (blurred) nematodes, the population of nematodes that are the detection targets can be recognized more accurately. Furthermore, even if the detection target image is acquired using a relatively inexpensive and simple microscope provided in a school or the like, and the image quality is significantly different from that of an image precisely acquired using a high-performance microscope or the like in a research institute, the population can be counted with high accuracy.

### Industrial Applicability

This invention is applicable to a determination as to whether to contain a specific substance, by taking advantage of the properties of nematodes.

### Reference Signs List

1 image recognition device
11 control unit
12 storage unit
120 detection target image database
121 single-body pattern image database
122 multiple-bodies pattern image database
123 noise image database
124 model image database
125 image acquisition program
126 multiple-bodies pattern image creation program
127 model image creation program
128 learning program
129 counting program
13 image acquisition unit
14 display unit
15 input unit
16 communication unit
17 liquid amount estimation unit
18 liquid supply unit
19 program storage medium
2 nematode trap plate
21 container
22 solid phase
23A, 23B nematode trapping recess
230 test liquid
23 edge
232 peripheral wall
233 upper edge
234 liquid level
3 nematode(s)
41 single-body pattern image
42 label “2” pattern image
43 label “3” pattern image
44 label “4” pattern image
45 noise image
5 model image
6 image recognition result image

1. A non-temporary tangible storage medium storing an image recognition program for causing a computer to function as:
detection target image acquisition means for acquiring a detection target image showing a plurality of detection targets;
extraction means for extracting from the detection target image a detection target presence area that possibly includes an image of the detection targets;
storage means for storing a plurality of pattern images including a single-body pattern image showing one of the detection targets and a multiple-bodies pattern image corresponding to an image showing two or more of the detection targets overlapping each other; and
recognition means for recognizing the population of detection targets included in the detection target presence area by detecting a degree of concordance between an image of the detection target presence area and each of the pattern images.

2. The storage medium according to claim 1, wherein the storage means stores a plurality of the single-body pattern images, and the image recognition program causes the computer to function as multiple-bodies pattern image creation means for creating the multiple-bodies pattern image by combining the plurality of the single-body pattern images.

3. The storage medium according to claim 2, wherein the multiple-bodies pattern image creation means creates the multiple-bodies pattern image by subjecting one or more single-body pattern images among the plurality of the single-body pattern images to a change in at least one of a position in a planar direction or a rotation angle on a plane.

4. A model image creation device for image recognition learning, comprising:
a single-body pattern image acquisition unit that acquires a single-body pattern image showing one of detection targets;
a multiple-bodies pattern image acquisition unit that acquires a multiple-bodies pattern image corresponding to an image showing two or more of the detection targets overlapping each other;
a noise image acquisition unit that acquires a noise image showing none of the detection targets and including noise; and
a model image creation unit that creates a model image by compositing the single-body pattern image, the multiple-bodies pattern image, and the noise image,
wherein the multiple-bodies pattern image is created by superimposing a plurality of the single-body pattern images among which one or more single-body pattern images is subjected to a change in at least one of a position in a planar direction or a rotation angle on a plane, and the noise image corresponds to an image showing at least one of an edge of a recess in which the detection targets are placed and a substance except the detection targets, the substance being contained in a liquid in the recess.

5. The model image creation device for image recognition learning according to claim 4, wherein the model image creation unit creates a model image by compositing at least the single-body pattern image, a label “2” pattern image corresponding to the multiple-bodies pattern image showing two of the detection targets overlapping each other, a label “multiple” pattern image corresponding to the multiple-bodies pattern image showing three or more of the detection targets overlapping each other, and the noise image.

6. An image recognition device comprising:
a detection target image acquisition unit that acquires a detection target image showing a plurality of detection targets;
an extraction unit that extracts from the detection target image a detection target presence area that possibly includes an image of the detection targets;
a storage unit that stores a plurality of pattern images including a single-body pattern image showing one of the detection targets and a multiple-bodies pattern image corresponding to an image showing two or more of the detection targets overlapping each other; and
a recognition unit that recognizes the population of detection targets included in the detection target presence area by detecting a degree of concordance between an image of the detection target presence area and each of the pattern images.

7. A detection target population counting method comprising:
acquiring, by detection target image acquisition means, a detection target image showing a plurality of detection targets;
extracting, by extraction means, from the detection target image a detection target presence area that possibly includes an image of the detection targets;
storing, by storage means, a plurality of pattern images including a single-body pattern image showing one of the detection targets and a multiple-bodies pattern image corresponding to an image showing two or more of the detection targets overlapping each other; and
counting, by a recognition unit, the population of detection targets included in the detection target presence area by detecting a degree of concordance between an image of the detection target presence area and each of the pattern images.

8. The detection target population counting method according to claim 7, wherein the detection targets are nematodes that swim by alternately repeating flexion and extension in a liquid recess in which a liquid is present, and the detection target image is captured in a state adjusted for image capture, in which a difference between a height of a liquid level of the liquid in the liquid recess and a height of an upper edge of a peripheral wall of the liquid recess falls within half a height that the liquid can rise above the upper edge.

9. The detection target population counting method according to claim 8, wherein before acquiring the detection target image by the detection target image acquisition means, an amount of the liquid with which the liquid recess is refilled, in order to bring the amount of the liquid into the state adjusted for image capture is estimated, and the liquid recess is refilled with the liquid by the estimated amount to bring the amount of the liquid into the state adjusted for image capture.

10. The detection target population counting method according to claim 8, wherein the detection target image shows an inside of the liquid recess in which the nematodes are present, and 0 to 100 nematodes are present per 0.01 mm² of an opening area of the liquid recess when viewed from an upper side of the liquid recess.

11. The detection target population counting method according to claim 8, wherein the detection target image is captured in a state in which two or more of the nematodes are extended.

12. The detection target population counting method according to claim 9, wherein the detection target image shows an inside of the liquid recess in which the nematodes are present, and 0 to 100 nematodes are present per 0.01 mm² of an opening area of the liquid recess when viewed from an upper side of the liquid recess.

13. The detection target population counting method according to claim 9, wherein the detection target image is captured in a state in which two or more of the nematodes are extended.

14. The detection target population counting method according to claim 10, wherein the detection target image is captured in a state in which two or more of the nematodes are extended.
